(19) United States
(12) Patent Application Publication
(10) Pub. No.: US 2025/0101499 A1
(43) Pub. Date: Mar. 27, 2025

(54) ANALYSIS OF RNA MOLECULES USING CATALYTIC NUCLEIC ACIDS

(71) Applicants: BioNTech SE, Mainz (DE); Rheinische Friedrich-Wilhelms-Universität Bonn, Bonn (DE)

(72) Inventors: Irena VLATKOVIC, Göttingen (DE); Katalin KARIKO, Mainz (DE); Janos LUDWIG, Mainz (DE)

(21) Appl. No.: 18/730,527

(22) PCT Filed: Jan. 19, 2023
(86) PCT No.: PCT/EP2023/051271

§ 371 Date: Jul. 19, 2024

(30) Foreign Application Priority Data
Jan. 21, 2022 (WO) PCT/EP2022/051409

(51) Int. Cl. C12Q 1/6823; C12Q 1/6837

(52) U.S. Cl. CPC C12Q 1/6823; C12Q 1/6837

(57) ABSTRACT

The present invention relates to a method for analyzing the structure of 5′ terminus of an RNA molecule in a population of RNA molecules using catalytic nucleic acids, e.g., for determining the presence or absence of a 5′ cap structure.

The present invention relates to a method for analyzing a population of RNA molecules, wherein the amount of RNA molecules having a 5′ cap structure is determined. Other aspects of the invention relate to a method for determining capping efficiency in a population of RNA molecules and a method for capped RNA synthesis quality control.

### BACKGROUND

In vitro-transcribed (IVT) mRNA-based therapeutics are emerging as novel biologics with a variety of applications, including recent vaccines against coronavirus disease 2019 (COVID-19) caused by severe acute respiratory syndrome coronavirus 2 (SARS-COV-2), and other infectious disease vaccine candidates, cancer immunotherapies, gene-editing treatments and protein replacement therapies [1-6]. Ongoing preclinical and clinical studies using IVT mRNAs require innovative solutions for quality control of the IVT mRNA.

The presence of the cap structure at its 5′terminus is one of the key features of IVT mRNA that increases its stability and translatability. Capped mRNAs are generally translated more efficiently compared to uncapped mRNAs [7-9]. After synthesis, enzymatic and co-transcriptional capping of IVT mRNA may be incomplete, leading to the presence of a variable number of uncapped molecules in the final IVT mRNA.

Current approaches for determining the capping efficiency of IVT mRNA have limited applicability. In one method, IVT mRNA is transcribed from a short DNA template in the presence of [-32P] GTP and a cap analog. Digestion of this generated IVT mRNA with RNase T2 has been shown to release radioactive cap structure containing [³²P]-labeled 3′-phosphate by using anion exchange chromatography and subsequent measurements [10, 11]. However, this approach is only applicable for RNAs <50 nt long. Others have determined the efficiency of enzymatic capping of long mRNA transcribed in the presence of [γ-32P] GTP by measuring the reduction of mRNA radioactivity, as successful capping eliminates the labeled gamma phosphate [12]. The major disadvantage of these methods is the use of radioactive material for mRNA production.

In another approach, the capping efficiency of IVT mRNAs of different lengths (>1,000 nt) was measured without the use of radioactivity. In this procedure, a complementary biotin-tagged oligonucleotide was annealed to the 5′-end of the IVT mRNA which was then cleaved by RNase H [13]. The cleaved 5′-end was purified using streptavidin-coated magnetic beads and then analyzed by liquid chromatography and mass spectrometry (LC-MS). Although this method avoids radiolabeling of the mRNA, the RNase H cleavage site was not unique since additional cleavage products of different lengths were also generated, making the method not fully reliable. Furthermore, analysis of the cleaved fragments requires LC-MS, which impacts affordability of the assay.

Recently, a biosensor detecting both capping level and integrity of mRNA was developed [14]. However, the biosensor method only allows the detection of variations in capping level in increments of at least 20%, making this method difficult to use as a precise analytical tool.

### SUMMARY

An aspect of the invention is directed to a method for analyzing a population of RNA molecules, said method comprising the steps
(a) contacting a catalytic nucleic acid molecule with a population of RNA molecules, which population comprises one or more RNA molecules comprising a cleavage site for the catalytic nucleic acid molecule and a 5′ cap structure, under conditions allowing the cleavage of the RNA molecules to produce a 5′ terminal fragment and at least one 3′ fragment,
(b) separating the 5′ terminal fragment obtained in step (a) at least partially from the at least one 3′ fragment, resulting in a population of 5′ terminal fragments, and
(c) determining in the population of 5′ terminal fragments obtained in step (b) the amount of RNA molecules having the 5′ cap structure.

An aspect of the invention is directed to a method for determining capping efficiency in a population of RNA molecules, said method comprising the steps:
(a) contacting a catalytic nucleic acid molecule with a population of RNA molecules, which population comprises one or more RNA molecules comprising a cleavage site for the catalytic nucleic acid molecule and a 5′ cap structure, under conditions allowing the cleavage of the RNA molecules to produce a 5′ terminal fragment and at least one 3′ fragment,
(b) separating the 5′ terminal fragment obtained in step (a) at least partially from the at least one 3′ fragment, resulting in a population of 5′ terminal fragments, and
(c) determining in the population of 5′ terminal fragments obtained in step (b) the amount of RNA molecules having the 5′ cap structure.

An aspect of the invention is directed to a method for analyzing an RNA molecule, comprising the steps:
(i) synthesizing an RNA molecule,
(ii) capping the RNA synthesized in (i), and
(iii) analyzing the RNA molecule by the method for analyzing a population of RNA molecules of the invention.

An aspect of the invention is directed to a method for capped RNA synthesis quality control, comprising the steps:
(i) synthesizing an RNA molecule,
(ii) capping the RNA synthesized in (i), and
(iii) analyzing the RNA molecule by the method for analyzing a population of RNA molecules of the invention.

An aspect of the invention is directed to a catalytic nucleic acid molecule,
(i) a sequence selected from SEQ ID NO: 1-25,
(ii) a sequence having at least 80% identity with any one of SEQ ID NO: 1-25, and/or
(iii) a fragment of (i) and/or (ii), wherein the catalytic nucleic acid molecule comprises a catalytic core.

An aspect of the invention is directed to a use of the catalytic nucleic acid molecule as described herein in the method for analyzing a population of RNA molecules, in a method for determining capping efficiency in a population of RNA molecules, in a method for analyzing an RNA molecule, and/or a method for capped RNA synthesis quality control.

The step of separating the 5′ terminal fragment at least partially from the 3′ fragment(s) prior to the step of determining the amount of RNA molecules having the 5′ cap structure is to provide for a population that is enriched (at least partially purified) for 5′ terminal fragments, i.e., the quantity of 5′ terminal fragments as a percentage of all cleaved and uncleaved RNA molecules present in the separated (or purified) enriched population obtained after the separation step is greater than the quantity of 5′ terminal fragments as a percentage of all cleaved and uncleaved RNA molecules present in the population prior to the separation step. This enriched or partially purified population is then subjected to the step of determining the amount of RNA molecules having a 5′ cap structure. By first providing for this enriched or partially purified population, the sensitivity and accuracy of the assay in increased.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise.

The term “about” means approximately or nearly, and in the context of a numerical value or range set forth herein preferably means+/−10% of the numerical value or range recited or claimed.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless expressly specified otherwise, the term “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e., for the purpose of this embodiment “comprising” is to be understood as having the meaning of “consisting of”.

Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present invention was not entitled to antedate such disclosure.

The presence of the cap structure on the 5′-end of in vitro-transcribed (IVT) mRNA determines its translation and stability, underpinning its use in therapeutics. Both enzymatic and co-transcriptional capping may lead to incomplete positioning of the cap on newly synthesized RNA molecules. IVT mRNAs are rapidly emerging as novel biologics, including recent vaccines against COVID-19 and vaccine candidates against other infectious diseases as well as for cancer immunotherapies and protein replacement therapies. Quality control methods necessary for the preclinical and clinical stages of development of these therapeutics are under ongoing development.

Capped mRNAs are generally translated more efficiently compared to uncapped mRNAs [7-9]. After synthesis, enzymatic and co-transcriptional capping of IVT mRNA may be incomplete, leading to the presence of a variable number of uncapped molecules in the final IVT mRNA. The inventors developed methods that allow fast and simple quantitative measurements of the capping efficiency. These methods can be used as a quality control for mRNA-based therapeutics.

In the Examples of the invention, catalytic nucleic acid molecules (in particular ribozymes, Rz) were designed to specifically cleave IVT mRNA at a unique position in close proximity to the 5′-end, releasing capped or uncapped short 5′ cleavage products in a range of 10-30 nt. The well-defined capped or uncapped 5′ cleavage products cut off by the ribozyme differ in length from each other by one nucleoside in length, specifically the cap structure itself. The difference in length will be greater than one nucleotide where the cap structure is greater than one nucleotide in length.

In comparison to other methodologies for analyzing the 5′ end of an RNA molecule, the methods of the invention are improved in that they are more sensitive, more accurate and allow for higher reproducibility of results, which improvements make the methods especially applicable for quality control of mRNA-based therapeutics.

An aspect of the invention is directed to a method for analyzing a population of RNA molecules, said method comprising the steps
(a) contacting a catalytic nucleic acid molecule with a population of RNA molecules, which population comprises one or more RNA molecules comprising a cleavage site for the catalytic nucleic acid molecule and a 5′ cap structure under conditions allowing the cleavage of the RNA molecules to produce a 5′ terminal fragment and at least one 3′ fragment,
(b) separating the 5′ terminal fragment obtained in step (a) at least partially from the at least one 3′ fragment, resulting in a population of 5′ terminal fragments, and
(c) determining in the population of 5′ terminal fragments obtained in step (b) the amount of RNA molecules having the 5′ cap structure.

In some embodiments, the methods are performed in the order of the steps specified.

The term “nucleic acid” according to the invention also comprises a chemical derivatization of a nucleic acid on a nucleotide base, on the sugar or on the phosphate, and nucleic acids containing non-natural nucleotides and nucleotide analogs. In some embodiments, the nucleic acid is a deoxyribonucleic acid (DNA) or a ribonucleic acid (RNA). In general, a nucleic acid molecule or a nucleic acid sequence refers to a nucleic acid which is preferably deoxyribonucleic acid (DNA) or ribonucleic acid (RNA). According to the invention, nucleic acids comprise genomic DNA, cDNA, mRNA, viral RNA, recombinantly prepared and chemically synthesized molecules. According to the invention, a nucleic acid may be in the form of a single-stranded or double-stranded and linear or covalently closed circular molecule.

According to the invention “nucleic acid sequence” refers to the sequence of nucleotides in a nucleic acid, e.g.; a ribonucleic acid (RNA) or a deoxyribonucleic acid (DNA). The term may refer to an entire nucleic acid molecule (such as to the single strand of an entire nucleic acid molecule) or to a part (e.g. a fragment) thereof.

According to the present invention, the term “RNA” or “RNA molecule” relates to a molecule which comprises ribonucleotide residues and which is preferably entirely or substantially composed of ribonucleotide residues. The term “ribonucleotide” relates to a nucleotide with a hydroxyl group at the 2′-position of a β-D-ribofuranosyl group. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of an RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally occurring RNAs.

According to the invention, RNA may be single-stranded or double-stranded. In some embodiments of the present invention, single-stranded RNA is preferred. The term “single-stranded RNA” generally refers to an RNA molecule to which no complementary nucleic acid molecule (typically no complementary RNA molecule) is associated. Single-stranded RNA may contain self-complementary sequences that allow parts of the RNA to fold back and to form secondary structure motifs including without limitation base pairs, stems, stem loops and bulges. Single-stranded RNA can exist as minus strand [(−) strand] or as plus strand [(+) strand]. The (+) strand is the strand that comprises or encodes genetic information. The genetic information may be for example a polynucleotide sequence encoding a protein. When the (+) strand RNA encodes a protein, the (+) strand may serve directly as template for translation (protein synthesis). The (−) strand is the complement of the (+) strand. In the case of double-stranded RNA, (+) strand and (−) strand are two separate RNA molecules, and both these RNA molecules associate with each other to form a double-stranded RNA (“duplex RNA”).

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

The term “population of RNA molecules” relates to a plurality of RNA molecules. The population can be mixture of more than one RNA molecule species, or can be a single species of RNA molecules. The RNA molecules of a species of RNA molecules may have essentially the same nucleotide sequence. The population of RNA molecules can be obtained from any natural source, or can be obtained by synthesis. Any synthesis method known to the skilled person may be used. RNA synthesis may include in-vitro transcription (IVT) or solid-phase synthesis. RNA synthesis may also include transcription and replication in a cellular system. Synthesis includes capping of an RNA molecule, if not indicated herein as a separate step in the manufacture of the RNA molecule. A “species of RNA molecules” can include a capped RNA molecule and a non-capped RNA molecule.

According to the present invention, “RNA replication” generally refers to an RNA molecule synthesized based on the nucleotide sequence of a given RNA molecule (template RNA molecule). The RNA molecule that is synthesized may be, e.g., identical or complementary to the template RNA molecule. In general, RNA replication may occur via synthesis of a DNA intermediate, or may occur directly by RNA-dependent RNA replication mediated by an RNA-dependent RNA polymerase (RdRP).

The RNA molecule may comprise a translatable nucleic acid sequence, i.e. translatable into a peptide or protein. For an RNA sequence to be “translatable”, it requires the presence of a 5′ cap structure.

“3′ end of a nucleic acid” refers according to the invention to that end which has a free hydroxy group. In a diagrammatic representation of double-stranded nucleic acids, in particular DNA, the 3′ end is always on the right-hand side. “5′ end of a nucleic acid” refers according to the invention to that end which has a free phosphate group. In a diagrammatic representation of double-strand nucleic acids, in particular DNA, the 5′ end is always on the left-hand side.

| | | |
|---|---|---|
| 5′ end | 5′--P-NNNNNNN-OH-3′ | 3′ end |
| | 3′-HO-NNNNNNN-P--5′ | |

“5′ terminal fragment” of a nucleic acid molecule relates to a fragment of the nucleic acid molecule, including the 5′ terminal sequence of the nucleic acid molecule.

“3′ fragment” of a nucleic acid molecule relates to any fragment of the nucleic acid molecule including a sequence of the nucleic acid molecule downstream the sequence of the 5′ terminal fragment.

“3′ terminal fragment” of a nucleic acid molecule is a 3′ fragment including the 3′ terminal sequence of the nucleic acid molecule.

In some embodiments, the 5′ terminal fragment and the 3′ fragment of a nucleic acid molecule, in particular an RNA molecule, can be obtained by cleavage with a catalytic nucleic acid molecule, resulting in a 5′ terminal fragment and at least one 3′ fragment. Cleavage at a single cleavage site results in one 5′ terminal fragment and one 3′ fragment (i.e. a 3′ terminal fragment). Cleavage at two or more cleavage sites results in one 5′ terminal fragment and two or more 3′ fragments.

In some embodiments, the sequences of the 5′ terminal fragment and at least one 3′ terminal fragments do not overlap with each other.

Any RNA population comprising one or more RNA molecules having a 5′ cap structure may be subjected to the method as described herein. In some embodiments, the population of RNA molecules can be a population of mRNA molecules, self-replicating RNA, ncRNA and/or snRNA.

According to the invention, the term “mRNA” means “messenger-RNA” and relates to a transcript which is typically generated by using a DNA template and encodes a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to the skilled person. For example, there is a variety of in vitro transcription kits commercially available. According to the invention, mRNA may be modified by stabilizing modifications and capping.

According to the invention, the terms “poly(A) sequence” or “poly(A) tail” refer to an uninterrupted or interrupted sequence of adenylate residues which is typically located at the 3′ end of an RNA molecule. An uninterrupted sequence is characterized by consecutive adenylate residues. In nature, an uninterrupted poly(A) sequence is typical. A poly(A) sequence is normally not encoded by eukaryotic DNA, but is attached during eukaryotic transcription in the cell nucleus to the free 3′ end of the RNA by a template-independent RNA polymerase after transcription, but a poly(A) sequence can also be encoded by DNA. Thus, RNA molecules can encompass poly(A) sequences both encoded by DNA and/or enzymatically polyadenylated.

According to the invention, the term “self-replicating RNA” includes any RNA capable of replicating autonomously in a host cell. “Self-replicating RNA” include an RNA virus, which may have a single-stranded RNA (ssRNA) genome and include alphaviruses, flaviviruses, measles viruses (MVs) and rhabdoviruses. Alphaviruses and flaviviruses possess a genome of positive polarity, whereas the genome of measles viruses (MVs) and rhabdoviruses is negative strand ssRNA. Typically, a self-replicating virus is a virus with a (+) stranded RNA genome which can be directly translated after infection of a cell, and this translation provides an RNA-dependent RNA polymerase which then produces both antisense and sense transcripts from the infected RNA. In some embodiments, self-replicating RNA can be a type of mRNA.

Non-coding RNA (ncRNA) relates to an RNA molecule that is not translated into a polypeptide or protein.

Small nuclear RNA (snRNA) relates to a class of small RNA molecules that are found within the nucleus of eukaryotic cells.

In some embodiments, in step (a) the catalytic nucleic acid molecule may be contacted with a population of RNA molecules obtained by in-vitro transcription or solid-phase synthesis.

The terms “transcription” and “transcribing” relate to a process during which a nucleic acid molecule with a particular nucleic acid sequence (the “nucleic acid template”) is read by an RNA polymerase so that the RNA polymerase produces a single-stranded RNA molecule. During transcription, the genetic information in a nucleic acid template is transcribed. The nucleic acid template may be DNA; however, e.g.; in the case of transcription from an RNA viral nucleic acid template, the template is typically RNA. Subsequently, the transcribed RNA may be translated into protein. According to the present invention, the term “transcription” comprises “in vitro transcription (IVT)”, wherein the term “in vitro transcription” relates to a process wherein RNA, in particular mRNA, is in vitro synthesized in a cell-free system. Preferably, cloning vectors are applied for the generation of transcripts. These cloning vectors are generally designated as transcription vectors and are according to the present invention encompassed by the term “vector”. The cloning vectors are preferably plasmids. According to the present invention, RNA preferably is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

The in vitro transcription methodology is known to the skilled person. For example, as mentioned in WO 2011/015347 A1, a variety of in vitro transcription kits is commercially available.

The single-stranded nucleic acid molecule produced during transcription typically has a nucleic acid sequence that is the complementary sequence of the template.

According to the invention, the terms “template” or “nucleic acid template” or “template nucleic acid” generally refer to a nucleic acid sequence that may be replicated or transcribed.

“Nucleic acid sequence transcribed from a nucleic acid sequence” and similar terms refer to a nucleic acid sequence, where appropriate as part of a complete RNA molecule, which is a transcription product of a template nucleic acid sequence. Typically, the transcribed nucleic acid sequence is a single-stranded RNA molecule.

The term “vector” is used here in its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, virus genomes, and fractions thereof.

“Solid phase synthesis” of a nucleic acid molecule, in particular an RNA molecule, refers to chemical synthesis of a nucleic acid molecule, using nucleotide or oligonucleotide building blocks. Solid phase synthesis of a nucleic acid molecule is known to the skilled person. For example, phosphoramidite chemistry may be used.

In some embodiments, in the RNA molecule, the cleavage site may be located at least 5 nt, at least 10 nt, or at least 15 nt downstream the 5′ end of the RNA molecule.

In some embodiments, in the RNA molecule, the cleavage site may be located at most 50 nt or at most 35 nt downstream the 5′ end of the RNA molecule.

In some embodiments, the cleavage site may be located in a range of 5-50 nucleotides downstream the 5′ end of the RNA molecule.

In some embodiments, the cleavage site may also be located in a range of 10-50 nucleotides downstream the 5′ end of the RNA molecule.

In some embodiments, the cleavage site may also be located in a range of 15-30 nucleotides downstream the 5′ end of the RNA molecule.

In some embodiments, the RNA molecule may comprise a 5′ UTR sequence. Suitable 5′ UTR sequences are known to the skilled person. For example, the 5′ UTR can be selected form Human alpha globin (hAg) 5′ UTR and tobacco etch virus (TEV) 5′ UTR.

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR).

A 3′-UTR, if present, is located at the 3′ end of a gene, downstream of the termination codon of a protein-encoding region, but the term “3′-UTR” does preferably not include the poly(A) tail. Thus, the 3′-UTR is upstream of the poly(A) tail (if present), e.g. directly adjacent to the poly(A) tail.

A 5′-UTR, if present, is located at the 5′ end of a gene, upstream of the start codon of a protein-encoding region. A 5′-UTR is downstream of the 5′-cap (if present), e.g. directly adjacent to the 5′-cap.

5′- and/or 3′-untranslated regions may, according to the invention, be functionally linked to an open reading frame, so as for these regions to be associated with the open reading frame in such a way that the stability and/or translation efficiency of the RNA comprising said open reading frame are increased.

UTRs are implicated in stability and translation efficiency of RNA, e.g., mRNA. Both can be improved, besides structural modifications concerning the 5′-cap and/or the 3′ poly(A)-tail as described herein, by selecting specific 5′ and/or 3′ untranslated regions (UTRs). Sequence elements within the UTRs are generally understood to influence translational efficiency (mainly 5′-UTR) and RNA stability (mainly 3′-UTR). It is preferable that a 5′-UTR is present that is active in order to increase the translation efficiency and/or stability of the RNA replicon. Independently or additionally, it is preferable that a 3′-UTR is present that is active in order to increase the translation efficiency and/or stability of the RNA replicon.

A 5′-UTR according to the present invention can comprise any combination of more than one nucleic acid sequence, optionally separated by a linker. A 3′-UTR according to the present invention can comprise any combination of more than one nucleic acid sequence, optionally separated by a linker.

The term “linker” according to the invention relates to a nucleic acid sequence added between two nucleic acid sequences to connect said two nucleic acid sequences. There is no particular limitation regarding the linker sequence.

A 3′-UTR typically has a length of 200 to 2000 nucleotides, e.g. 500 to 1500 nucleotides. The 3′-untranslated regions of immunoglobulin mRNAs are relatively short (fewer than about 300 nucleotides), while the 3′-untranslated regions of other genes are relatively long. For example, the 3′-untranslated region of tPA is about 800 nucleotides in length, that of factor VIII is about 1800 nucleotides in length and that of erythropoietin is about 560 nucleotides in length. The 3′-untranslated regions of mammalian mRNA typically have a homology region known as the AAUAAA hexanucleotide sequence. This sequence is presumably the poly(A) attachment signal and is frequently located from 10 to 30 bases upstream of the poly(A) attachment site. 3′-untranslated regions may contain one or more inverted repeats which can fold to give stem-loop structures which act as barriers for exoribonucleases or interact with proteins known to increase RNA stability (e.g. RNA-binding proteins).

In some embodiments, the RNA molecule can comprise at least one cleavage site for the catalytic nucleic acid molecule. In some embodiments, the RNA molecule has only one site that can be cleaved by the catalytic nucleic acid used in step (a).

In some embodiments, the catalytic nucleic acid may specifically cleave the RNA molecule at the at least one cleavage site. Specific cleavage at a single cleavage site results in one 5′ terminal fragment and one 3′ fragment. Specific cleavage at two or more cleavage sites results in one 5′ terminal fragment and two or more 3′ fragments.

In some embodiments, the RNA molecule can comprise one cleavage site for the catalytic nucleic acid molecule. The cleavage site may be present in a range of 5-50 nucleotides downstream the 5′ end of the RNA molecule, 10-50 nucleotides downstream the 5′ end of the RNA molecule, or 15-30 nucleotides downstream the 5′ end of the RNA molecule. The other cleave sites may be located downstream this range. For separation of the 3′ fragments from the 5′ terminal fragments, the at least one 3′ fragments may be longer than the 5′ terminal fragment. For example, the at least one 3′ fragment may have a length of 100, 200, 500 or even more nucleotides.

“Upstream” describes the relative positioning of a first element of a nucleic acid molecule with respect to a second element of that nucleic acid molecule, wherein both elements are comprised in the same nucleic acid molecule, and wherein the first element is located nearer to the 5′ end of the nucleic acid molecule than the second element of that nucleic acid molecule. The second element is then said to be “downstream” of the first element of that nucleic acid molecule. An element that is located “upstream” of a second element can be synonymously referred to as being located “5” of that second element. For a double-stranded nucleic acid molecule, indications like “upstream” and “downstream” are given with respect to the (+) strand.

In some embodiments, the catalytic nucleic acid molecule of the invention may cleave at a cleavage site in a 5′ UTR sequence. For example, a catalytic nucleic acid molecule comprising a sequence selected from SEQ ID NO:1-4 and SEQ ID NO:6-25 recognizes a sequence in the 5′ UTR of hAg, and a catalytic nucleic acid molecule comprising SEQ ID NO:5 recognizes a sequence in the 5′ UTR of TEV.

In some embodiments, the catalytic nucleic acid molecule may cleave at a 5′-NUH-3′ cleavage site in the RNA molecule to produce a 5′ fragment comprising a NUH>p 3′ end, wherein
N is selected from G, A, C and U; and
H is selected from A, C and U.

In some embodiments, the catalytic nucleic acid molecule may cleave at a 5′-NCH-3′ cleavage site in the RNA molecule to produce a 5′ fragment comprising a NCH>p 3′ end, wherein
N is selected from G, A, C and U; and
H is selected from A, C and U.

The NUH and NCH cleavage sites of catalytic nucleic acids are known to the skilled person.

As used herein, NUH>p 3′ and NCH>p 3′ indicate cleavage in the RNA molecule between the NUH or NCH sequence and the nucleotide directly 3′ adjacent to this sequence. For example, a catalytic nucleic acid molecule comprising SEQ ID NO:1, 3 or 4 cleaves 3′ the sequence GUC (Table 4 of Example 1). In another example, a catalytic nucleic acid molecule comprising SEQ ID NO:2 cleaves 3′ the sequence GUA. In a further example, a catalytic nucleic acid molecule comprising SEQ ID NO: 5 cleaves 3′ the sequence ACA. Examples of further cleavage sites are disclosed in Table 1.

A “catalytic nucleic acid molecule” or “catalytic nucleic acid” relates to a nucleic acid molecule having nucleic acid cleavage activity. The catalytic nucleic acid can comprise a “catalytic core sequence” or “catalytic core” and 3′ and 5′ flanking sequences.

In some embodiments the catalytic nucleic acid molecule may have a length of 30-60 nt or 35-50 nt. In some embodiments, a catalytic core may be 20-25 nt in length, preferably 22 or 23 nt.

In some embodiments a flanking sequence can comprise a “recognition sequence”, i.e., a sequence specifically recognizing a target sequence in an RNA molecule. In some embodiments the flanking sequences can be 5-20 nt in length.

In some embodiments the catalytic nucleic acid molecule may be a ribozyme or a DNAzyme. Ribozymes and DNAzymes are known to the skilled person. Design and construction of a catalytic nucleic acid molecule specifically binding to a target sequence in a nucleic acid molecule, and specifically cleaving the nucleic acid molecule, are known to the skilled person.

“Ribozyme” relates to an antisense RNA molecule that combines ability to recognize a specific target on RNA substrate and cleave that target by acid-base catalysis. There are different types of ribozymes of which the well-studied and often applied is hammerhead. Hammerhead RNA cleavage reaction is a phosphodiester isomerization from a 5′ to 3′ diester to a 2′,3′-cyclic phosphate diester, resulting in the cleavage of the phosphate backbone. While first ribozymes were found in nature as RNAS able to catalyze a self-cleavage, in 1988, Haseloff & Gerlach (https://www.nature.com/articles/334585a0.pdf) separated their substrate and nuclease activities. They proposed a model for design and synthesis of ribozymes and proposed a minimal structural requirements for ribozyme-catalyzed RNA cleavage: (A) contains conserved sequences in the RNA substrate (cleavage sites e.g. GUC and other triplets) immediately adjacent to the site of cleavage (B) comprises the naturally highly conserved sequences maintained in the ribozyme (core sequence), and the regions (C) consist of flanking helices, with base-pairing between the substrate and ribozyme RNAs (that allow accurate positioning of the ribozyme on the substrate RNA and stable structure formation).

Examples of trans cleaving ribozymes are described in U.S. Pat. No. 6,656,731.

In some embodiments the catalytic nucleic acid molecule may be a hammerhead ribozyme, a hairpin ribozyme, or a HDV ribozyme.

In some embodiments the catalytic nucleic acid molecule may be a modified catalytic nucleic acid molecule. In some embodiments, the RNA molecule to be analyzed by a method as described herein may be a modified RNA molecule. In some embodiments the catalytic nucleic acid molecule and/or the RNA molecule may comprise at least one modified nucleotide.

A modified RNA molecule and/or catalytic nucleic acid molecule, as defined herein may contain nucleotide analogues/modifications, e.g., backbone modifications, sugar modifications or base modifications. A backbone modification in connection with the present invention is a modification, in which phosphates of the backbone of the nucleotides contained in an RNA molecule and/or catalytic nucleic acid molecule as defined herein are chemically modified. A sugar modification in connection with the present invention is a chemical modification of the sugar of the nucleotides of the RNA molecule and/or catalytic nucleic acid molecule as defined herein. Furthermore, a base modification in connection with the present invention is a chemical modification of the base moiety of the nucleotides of the RNA molecule and/or catalytic nucleic acid molecule. In this context, nucleotide analogues or modifications are preferably selected from nucleotide analogues, which are applicable for transcription and/or translation.

Sugar Modifications: The modified nucleosides and nucleotides, which may be incorporated into a modified RNA molecule and/or catalytic nucleic acid molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH2CH2 O)nCH2CH2OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

Backbone Modifications: The phosphate backbone may further be modified in the modified nucleosides and nucleotides, which may be incorporated into a modified RNA molecule and/or catalytic nucleic acid molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications: The modified nucleosides and nucleotides, which may be incorporated into a modified RNA molecule and/or catalytic nucleic acid molecule as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particular embodiments of the present invention, the nucleotide analogues/modifications are selected from base modifications, which are preferably selected from 2-amino-6-chloropurineriboside-5′-triphosphate, 2-aminopurine-riboside-5′-triphosphate; 2-aminoadenosine-5′-triphosphate, 2′-amino-2′-deoxy-cytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thio-uridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deaza-adenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudo-uridine-5′-triphosphate, or puromycin-5′-triphosphate, xanthosine-5′-triphosphate. Particular preference may be given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thiouridine, I-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydro-pseudouridine, 2-thio-dihydrouridine, 2-thio-dihydropseudouridine, 2-methoxy-uridine, 2-methoxy-4-thio-uridine, 4-methoxy-pseudouridine, and 4-methoxy-2-thio pseudouridine.

In some embodiments, modified nucleosides include 5-aza-cytidine, pseudoisocytidine, 3-methylcytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methylcytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methylcytidine, 4-methoxy-pseudoisocytidine, and 4-methoxy-I-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methyl-thio-N6-threonylcarbamoyladenosine, N6, N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2, N2-dimethylguanosine, 8-oxo-guanosine, 7-methyl-8-oxo-guanosine, I-methyl-6-thio-guanosine, N2-methyl-6-thio-guanosine, and N2, N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-O—(I-thiophosphate)-adenosine, 5′-O—(I-thiophosphate)-cytidine, 5′-O—(I-thiophosphate)-guanosine, 5′-O—(I-thiophosphate)-uridine or 5′-O—(I-thiophosphate)-pseudouridine.

In further embodiments, a modified RNA molecule and/or catalytic nucleic acid molecule may comprise nucleoside modifications selected from 6-aza-cytidine, 2-thio-cytidine, a-thio-cytidine, pseudo-iso-cytidine, 5-aminoallyl-uridine, 5-iodo-uridine, N1-methyl-pseudouridine, 5,6-dihydrouridine, a-thio-uridine, 4-thio-uridine, 6-aza-uridine, 5-hydroxy-uridine, deoxy-thymidine, 5-methyl-uridine, pyrrolo-cytidine, inosine, a-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, a-thio-adenosine, 8-azido-adenosine, 7-deaza-adenosine.

In certain preferred embodiments, the RNA molecule and/or catalytic nucleic acid molecule comprises a modified nucleoside in place of at least one (e.g., every) uridine, except as provided herein.

The term “uracil,” as used herein, describes one of the nucleobases that can occur in the nucleic acid of RNA. The structure of uracil is:

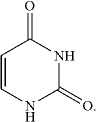

The term “uridine,” as used herein, describes one of the nucleosides that can occur in RNA. The structure of uridine is:

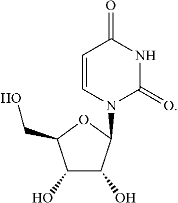 [SMILES: O=c1ccn([C@@H]2O[C@H](CO)[C@@H](O)[C@H]2O)c(=O)[nH]1]

UTP (uridine 5′-triphosphate) has the following structure:

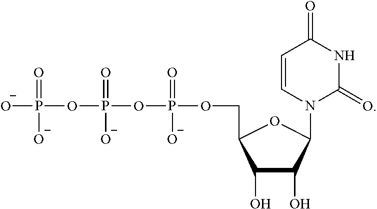

Modified uridines are also one of the nucleosides that can occur in RNA. One such modified uridine is pseudo-UTP (pseudouridine 5′-triphosphate) which has the following structure:

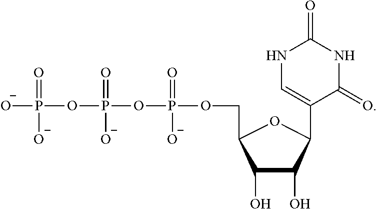

“Pseudouridine” is an exemplary modified nucleoside that is an isomer of uridine, where the uracil is attached to the pentose ring via a carbon-carbon bond instead of a nitrogen-carbon glycosidic bond. Another exemplary modified uridine is N1-methyl-pseudouridine (1mψ), which has the structure:

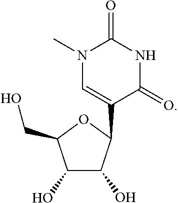 [SMILES: Cn1cc([C@@H]2O[C@H](CO)[C@@H](O)[C@H]2O)c(=O)[nH]c1=O]

N1-methyl-pseudo-UTP has the following structure:

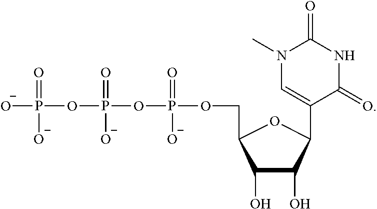

Another exemplary modified uridine is 5-methyl-uridine (m5U), which has the structure:

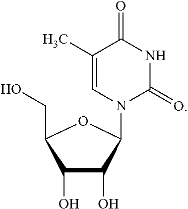

In certain preferred embodiments, one or more uridines in the RNA and/or catalytic nucleic acid molecule described herein is replaced by a modified nucleoside. In some embodiments, the modified nucleoside is a modified uridine.

In certain preferred embodiments, the RNA molecule and/or catalytic nucleic acid molecule comprises a modified uridine in place of at least one uridine. In some embodiments, the RNA molecule and/or catalytic nucleic acid molecule comprises a modified uridine in place of each uridine.

In certain preferred embodiments, the modified uridine is independently selected from pseudouridine (ψ), N1-methyl-pseudouridine (1mψ), and 5-methyl-uridine (m5U). In some embodiments, the modified uridine comprises pseudouridine (ψ). In some embodiments, the modified uridine comprises N1-methyl-pseudouridine (1mψ). In some embodiments, the modified uridine comprises 5-methyl-uridine (m5U). In some embodiments, the RNA molecule and/or catalytic nucleic acid molecule may comprise more than one type of modified uridine, and the modified uridines are independently selected from pseudouridine (ψ), N1-methyl-pseudouridine (1mψ), and 5-methyl-uridine (m5U). In some embodiments, the modified uridines comprise pseudouridine (ψ) and N1-methyl-pseudouridine (1mψ). In some embodiments, the modified uridines comprise pseudouridine (ψ) and 5-methyl-uridine (m5U). In some embodiments, the modified uridines comprise N1-methyl-pseudouridine (1 ml) and 5-methyl-uridine (m5U). In some embodiments, the modified uridines comprise pseudouridine (ψ), N1-methyl-pseudouridine (1 ml), and 5-methyl-uridine (m5U).

In certain preferred embodiments, the modified nucleoside replacing one or more, e.g., all, uridine in the RNA molecule and/or catalytic nucleic acid molecule may be any one or more of the following modified uridines: 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se2U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D) dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl)-pseudouridine (acp³ ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, or any other modified uridine known in the art.

In an embodiment, the RNA molecule and/or catalytic nucleic acid molecule comprises other modified nucleosides or comprises further modified nucleosides, e.g., modified cytidine such as those described above. For example, in one embodiment, in the RNA 5-methylcytidine is substituted partially or completely, preferably completely, for cytidine. In one embodiment, the RNA molecule and/or catalytic nucleic acid molecule comprises 5-methylcytidine and one or more selected from pseudouridine (ψ), N1-methyl-pseudouridine (1m), and 5-methyl-uridine (m⁵U). In an embodiment, the RNA molecule and/or catalytic nucleic acid molecule comprises 5-methylcytidine and N1-methyl-pseudouridine (1mψ). In some embodiments, the RNA molecule and/or catalytic nucleic acid molecule comprises 5-methylcytidine in place of each cytidine and N1-methyl-pseudouridine (1m) in place of each uridine.

In some embodiments the catalytic nucleic acid molecule and/or the RNA molecule may comprise at least one N1-Methylpseudouridine (m¹ψ) nucleotide.

In some embodiments the catalytic nucleic acid molecule and/or the RNA molecule may comprise at least one nucleoside selected from inosine, 2′-O-methyladenosine, 2′-O-methylguanosine, 2′-O-methyluridine, and 2′-O-methylcytidine.

In some embodiments the catalytic nucleic acid and/or the RNA molecule may comprise at least one deoxyribonucleotide.

In some embodiments the catalytic nucleic acid molecule may comprise
(i) a sequence selected from SEQ ID NO: 1-25,
(ii) a sequence having at least 80% identity, preferably at least 90%, 95%, 96%, 97%, 98%, or 99% identity with any one of SEQ ID NO: 1-25, and/or
(iii) a fragment of (i) and/or (ii),
wherein the catalytic nucleic acid molecule comprises a catalytic core.

TABLE 1
SEQ ID NO: 1-25

| | | | | Length of the | | |
|---|---|---|---|---|---|---|
| | Rz sequence (5′-3′) | | | recognition | | |
| | catalytic core | | Type | sequence | | |
| | sequences are underlined, | Site | of | (left-right)- | | SEQ |
| | the rest depends on | that | the | depends on | Targeted | ID |
| Name | target sequence-UTR | cleaves | site | UTR | 5′UTR | NO: |
| Rz1 | UGU GGG CUG AUG AGG CCG | GUC | NUH | 6-10 | hAg | 1 |
| | UGA GGC CGA AAC CAG AAG | | | | | |
| | AAU | | | | | |
| Rz2 | GGG GAC CAG AAG AAC UGA | GUA | NUH | 14-9 | hAg | 2 |
| | UGA GGC CGU GAG GCC GAA | | | | | |
| | ACUm AmGmUm UmCmGm | | | | | |
| Rz3 | UGU GGG CUG AUG AGG CCG | GUC | NUH | 6-6 | hAg | 3 |
| | UGA GGC CGA AAC CAG AA | | | | | |
| Rz4 | AGU CUG UGG GCU GAU GAG | GUC | NUH | 10-9 | hAg | 4 |
| | GCC GUG AGG CCG AAA CCA | | | | | |
| | GAA GAA | | | | | |
| Rz5 | GUA UAC UGA UGA GGC CGU | ACA | NCH | 5-20 | TEV | 5 |
| | GAG GCC GAA IUmUm | | | | | |
| | GmUmGmUmUmGm AmGmAm | | | | | |
| | CmUmAm GmUmUm UmAm | | | | | |
| Rz6 | ucugagucCUGAUGAGGCCGUGAGG | ACA | NCH | 8-6 | hAg | 6 |
| | CCGAAIugggga | | | | | |
| Rz7 | gagucugCUGAUGAGGCCGUGAGGC | CCA | NCH | 7-7 | hAg | 7 |
| | CGAAIgggacca | | | | | |
| Rz8 | gucugugCUGAUGAGGCCGUGAGGC | CCC | NCH | 7-7 | hAg | 8 |
| | CGAAIgaccaga | | | | | |
| Rz9 | agucuguCUGAUGAGGCCGUGAGGC | CCC | NCH | 7-7 | hAg | 9 |
| | CGAAIggaccag | | | | | |
| Rz10 | gucuguCUGAUGAGGCCGUGAGGCC | CCC | NCH | 6-7 | hAg | 10 |
| | GAAIggaccag | | | | | |
| Rz11 | gaauacuCUGAUGAGGCCGUGAGGC | ACU | NCH | 7-7 | hAg | 11 |
| | CGAAIuuuauuc | | | | | |
| Rz12 | gaccagaCUGAUGAGGCCGUGAGGC | UCU | NCH | 7-7 | hAg | 12 |
| | CGAAIaauacua | | | | | |
| Rz13 | ggggaccagaCUGAUGAGGCCGUGA | UCU | NCH | 10-10 | hAg | 13 |
| | GGCCGAAIaauacuaguu | | | | | |
| Rz14 | ggggaccCUGAUGAGGCCGUGAGGC | UCU | NCH | 7-7 | hAg | 14 |
| | CGAAIaagaaua | | | | | |
| Rz15 | uguggggaccCUGAUGAGGCCGUGA | UCU | NCH | 10-10 | hAg | 15 |
| | GGCCGAAIaagaauacua | | | | | |
| Rz16 | ucuguggCUGAUGAGGCCGUGAGGC | UCU | NCH | 7-7 | hAg | 16 |
| | CGAAIaccagaa | | | | | |
| Rz17 | gagucuguggCUGAUGAGGCCGUGA | UCU | NCH | 10-10 | hAg | 17 |
| | GGCCGAAIaccagaagaa | | | | | |
| Rz18 | ugagucugugCUGAUGAGGCCGUGA | CCC | NCH | 10-10 | hAg | 18 |
| | GGCCGAAIgaccagaaga | | | | | |
| Rz19 | cugagucuguCUGAUGAGGCCGUGA | CCC | NCH | 10-10 | hAg | 19 |
| | GGCCGAAIggaccagaag | | | | | |
| Rz20 | gucuguCUGAUGAGGCCGUGAGGCC | CCC | NCH | 6-10 | hAg | 20 |
| | GAAIggaccagaag | | | | | |
| Rz21 | ucugagucugCUGAUGAGGCCGUGA | CCA | NCH | 10-10 | hAg | 21 |
| | GGCCGAAIgggaccagaa | | | | | |
| Rz22 | cugagucCUGAUGAGGCCGUGAGGC | ACA | NCH | 7-7 | hAg | 22 |
| | CGAAIuggggac | | | | | |
| Rz23 | ucucugagucCUGAUGAGGCCGUGA | ACA | NCH | 10-10 | hAg | 23 |
| | GGCCGAAIuggggaccag | | | | | |
| Rz24 | cucucugCUGAUGAGGCCGUGAGGC | ACU | NCH | 7-7 | hAg | 24 |
| | CGAAIucugugg | | | | | |
| Rz25 | guucucucugCUGAUGAGGCCGUGA | ACU | NCH | 10-10 | hAg | 25 |
| | GGCCGAAIucugugggga | | | | | |

Hammerhead Rz catalytic core sequences are underlined;
hAg, human alpha globin;
I, inosine;
m, 2′-O-Met;
TEV, tobacco etch virus;
Rz, ribozyme;
5′UTR, 5′ untranslated region.

TABLE 2
catalytic core sequences of SEQ ID NO: 1-25

| SEQ | | |
|---|---|---|
| ID | (5′-3′) catalytic | Core sequence present |
| NO | core sequences | in SEQ ID NO(s): |
| 26 | CUGAUGAGGCCGUGAGGCCGAAA | 1-4 |
| 27 | CUGAUGAGGCCGUGAGGCCGAAI | 5-25 |

“Fragment”, with reference to a nucleic acid sequence, relates to a part of a nucleic acid sequence, i.e.; a sequence which represents the nucleic acid sequence shortened at the 5′- and/or 3′-end(s). Preferably, a fragment of a nucleic acid sequence comprises at least 80%, preferably at least 90%, 95%, 96%, 97%, 98%, or 99% of the nucleotide residues from said nucleic acid sequence. In the present invention those fragments of RNA molecules are preferred which retain RNA stability.

In some embodiments the fragment of a catalytic nucleic acid molecule, as described herein, can comprise the catalytic core of the catalytic nucleic acid molecules of any one of SEQ ID NOs: 1-25.

In some embodiments the catalytic core may comprise SEQ ID NO:26 or 27 (Table 2), or a sequence having at least 80% identity, preferably at least 90%, 95%, 96%, 97%, 98%, or 99% with SEQ ID NO: 26 and/or 27.

In some embodiments, in any one of SEQ ID NO: 1-25, Am can be independently selected from A and 2′-O-methyladenosine, Gm can be independently selected from G and 2′-O-methylguanosine, Um can be independently selected from U and 2′-O-methyluridine, and/or Cm can be independently selected from C and 2′-O-methylcytidine.

In some embodiments the catalytic nucleic acid molecule of the invention may be an RNA molecule, in particular a ribozyme.

The term “nucleic acid variants” include single or multiple nucleotide deletions, additions, mutations, substitutions and/or insertions in comparison with the reference nucleic acid. Deletions include removal of one or more nucleotides from the reference nucleic acid. Addition variants comprise 5′- and/or 3′-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations include abasic sites, crosslinked sites, and chemically altered or modified bases. Insertions include the addition of at least one nucleotide into the reference nucleic acid.

According to the invention, “nucleotide change” can refer to single or multiple nucleotide deletions, additions, mutations, substitutions and/or insertions in comparison with the reference nucleic acid. In some embodiments, a “nucleotide change” is selected from the group consisting of a deletion of a single nucleotide, the addition of a single nucleotide, the mutation of a single nucleotide, the substitution of a single nucleotide and/or the insertion of a single nucleotide, in comparison with the reference nucleic acid. According to the invention, a nucleic acid variant can comprise one or more nucleotide changes in comparison with the reference nucleic acid.

Variants of specific nucleic acid sequences preferably can have at least one functional property of said specific sequences and preferably are functionally equivalent to said specific sequences, e.g., nucleic acid sequences exhibiting properties identical or similar to those of the specific nucleic acid sequences.

Preferably the “degree of identity” or “identity” or “% identity” between a given nucleic acid sequence and a nucleic acid sequence which is a variant of said given nucleic acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence.

“Sequence identity” between two nucleic acid sequences indicates the percentage of nucleotides that are identical between the sequences.

The term “% identical” is intended to refer, in particular, to a percentage of nucleotides which are identical in an optimal alignment between two sequences to be compared, with said percentage being purely statistical, and the differences between the two sequences may be randomly distributed over the entire length of the sequence and the sequence to be compared may comprise additions or deletions in comparison with the reference sequence, in order to obtain optimal alignment between two sequences. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2:482, with the aid of the local homology algorithm by Needleman and Wunsch, 1970, J. Mol. Biol. 48:443, and with the aid of the similarity search algorithm by Pearson and Lipman, 1988, Proc. Natl Acad. Sci. USA 85:2444 or with the aid of computer programs using said algorithms (GAP, BESTFIT, FASTA, BLAST N and TFASTA in Wisconsin Genetics Software Package, Genetics Computer Group, 575 Science Drive, Madison, Wis.).

Percentage identity is obtained by determining the number of identical positions in which the sequences to be compared correspond, dividing this number by the number of positions compared and multiplying this result by 100.

For example, the BLAST program “BLAST 2 sequences” which is available on the website http://www.ncbi.nlm.nih.gov/blast/bl2seq/wblast2.cgi may be used.

“nt” is an abbreviation for nucleotide; or for nucleotides, preferably consecutive nucleotides in a nucleic acid molecule.

In some embodiments, in step (a) the catalytic nucleic acid molecule may be contacted with a population of RNA molecules capped by enzymatic or/and co-transcriptional capping in the presence of a capping analog.

The terms “5′-cap”, “cap”, “5′-cap structure”, “cap structure” are used synonymously and can refer to a dinucleotide that is found on the 5′ end of some eukaryotic primary transcripts such as precursor messenger RNA. A 5′-cap is a structure wherein a (optionally modified) guanosine is bonded to the first nucleotide of an mRNA molecule via a 5′ to 5′ triphosphate linkage (or modified triphosphate linkage in the case of certain cap analogs). The terms can refer to a conventional cap or to a cap analog.

“RNA which comprises a 5′-cap” or “RNA which is provided with a 5′-cap” or “RNA which is modified with a 5′-cap” or “capped RNA” refers to RNA which comprises a 5′-cap. For example, providing an RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

The term “conventional 5′-cap” refers to a naturally occurring 5′-cap, preferably to the 7-methylguanosine cap. In the 7-methylguanosine cap, the guanosine of the cap is a modified guanosine wherein the modification consists of a methylation at the 7-position.

In the context of the present invention, the term “5′-cap analog” refers to a molecular structure that resembles a conventional 5′-cap, but is modified to possess the ability to stabilize RNA if attached thereto, preferably in vivo and/or in a cell. A cap analog is not a conventional 5′-cap.

For the case of eukaryotic mRNA, the 5′-cap has been generally described to be involved in efficient translation of mRNA: in general, in eukaryotes, translation is initiated only at the 5′ end of a messenger RNA (mRNA) molecule, unless an internal ribosomal entry site (IRES) is present. Eukaryotic cells are capable of providing an RNA with a 5′-cap during transcription in the nucleus: newly synthesized mRNAs are usually modified with a 5′-cap structure, e.g.; when the transcript reaches a length of 20 to 30 nucleotides. First, the 5′ terminal nucleotide pppN (ppp representing triphosphate; N representing any nucleoside) is converted in the cell to 5′ GpppN by a capping enzyme having RNA 5′-triphosphatase and guanylyltransferase activities. The GpppN may subsequently be methylated in the cell by a second enzyme with (guanine-7)-methyltransferase activity to form the mono-methylated m⁷GpppN cap. In one embodiment, the 5′-cap used in the present invention is a natural 5′-cap.

In the present invention, a natural 5′-cap dinucleotide is typically selected from the group consisting of a non-methylated cap dinucleotide (G (5′)ppp(5′) N; also termed GpppN) and a methylated cap dinucleotide ((m⁷G (5′)ppp(5′) N; also termed m⁷GpppN). m⁷GpppN (wherein N is G) is represented by the following formula:

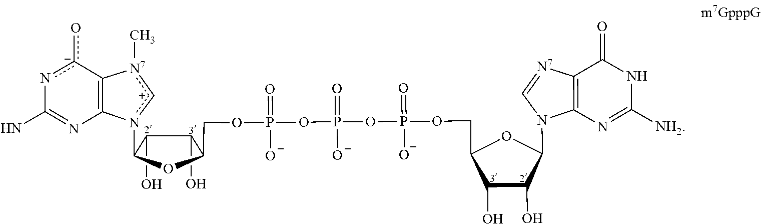

Capped RNA of the present invention can be prepared in vitro, and therefore, does not depend on a capping machinery in a host cell. The most frequently used method to make capped RNAs in vitro is to transcribe a DNA template with either a bacterial or bacteriophage RNA polymerase in the presence of all four ribonucleoside triphosphates and a cap dinucleotide such as m⁷G (5′)ppp(5′) G (also called m⁷GpppG). The RNA polymerase initiates transcription with a nucleophilic attack by the 3′-OH of the guanosine moiety of m⁷GpppG on the α-phosphate of the next templated nucleoside triphosphate (pppN), resulting in the intermediate m⁷GpppGpN (wherein N is the second base of the RNA molecule). The formation of the competing GTP-initiated product pppGpN is suppressed by setting the molar ratio of cap to GTP between 5 and 10 during in vitro transcription.

In preferred embodiments of the present invention, the 5′-cap (if present) is a 5′-cap analog. These embodiments are particularly suitable if the RNA is obtained by in vitro transcription, e.g. is an in vitro transcribed RNA (IVT-RNA). Cap analogs have been initially described to facilitate large scale synthesis of RNA transcripts by means of in vitro transcription.

For messenger RNA, some cap analogs (synthetic caps) have been generally described to date, and they can all be used in the context of the present invention. Ideally, a cap analog is selected that is associated with higher translation efficiency and/or increased resistance to in vivo degradation and/or increased resistance to in vitro degradation.

Preferably, a cap analog is used that can only be incorporated into an RNA chain in one orientation. Pasquinelli et al., 1995, RNA J. 1:957-967) demonstrated that during in vitro transcription, bacteriophage RNA polymerases use the 7-methylguanosine unit for initiation of transcription, whereby around 40-50% of the transcripts with cap possess the cap dinucleotide in a reverse orientation (i.e., the initial reaction product is Gpppm7GpN). Compared to the RNAs with a correct cap, RNAs with a reverse cap are not functional with respect to translation of a nucleic acid sequence into protein. Thus, it is desirable to incorporate the cap in the correct orientation, i.e., resulting in an RNA with a structure essentially corresponding to m⁷GpppGpN etc. It has been shown that the reverse integration of the cap-dinucleotide is inhibited by the substitution of either the 2′- or the 3′-OH group of the methylated guanosine unit (Stepinski et al., 2001, RNA J. 7:1486-1495; Peng et al., 2002, Org. Lett. 24:161-164). RNAs which are synthesized in presence of such “anti reverse cap analogs” are translated more efficiently than RNAs which are in vitro transcribed in presence of the conventional 5′-cap m⁷GpppG. To that end, one cap analog in which the 3′ OH group of the methylated guanosine unit is replaced by OCH₃ is described, e.g., by Holtkamp et al., 2006, Blood 108:4009-4017 (7-methyl (3′-O-methyl) GpppG; anti-reverse cap analog (ARCA)). ARCA is a suitable cap dinucleotide according to the present invention.

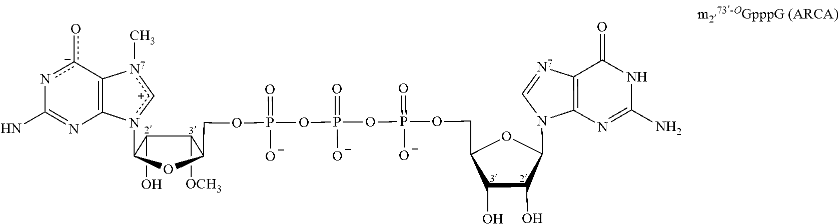

In an embodiment, the RNA of the present invention is essentially not susceptible to decapping. This is important because, in general, the amount of protein produced from synthetic mRNAs introduced into cultured mammalian cells is limited by the natural degradation of mRNA. One in vivo pathway for mRNA degradation begins with the removal of the mRNA cap. This removal is catalyzed by a heterodimeric pyrophosphatase, which contains a regulatory subunit (Dcp1) and a catalytic subunit (Dcp2). The catalytic subunit cleaves between the α and β phosphate groups of the triphosphate bridge. In the present invention, a cap analog may be selected or present that is not susceptible, or less susceptible, to that type of cleavage. A suitable cap analog for this purpose may be selected from a cap dinucleotide according to formula (I):

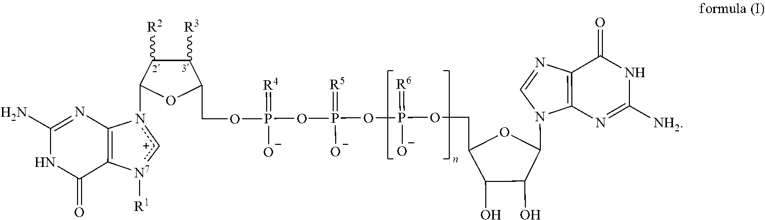

wherein R¹ is selected from the group consisting of optionally substituted alkyl, optionally substituted alkenyl, optionally substituted alkynyl, optionally substituted cycloalkyl, optionally substituted heterocyclyl, optionally substituted aryl, and optionally substituted heteroaryl,
R² and R³ are independently selected from the group consisting of H, halo, OH, and optionally substituted alkoxy, or R² and R³ together form O—X—O, wherein X is selected from the group consisting of optionally substituted CH₂, CH₂CH₂, CH₂CH₂CH₂, CH₂CH(CH₃), and
C(CH₃)₂, or R² is combined with the hydrogen atom at position 4′ of the ring to which R² is attached to form —O—CH₂— or —CH₂—O—,
R⁵ is selected from the group consisting of S, Se, and BH₃,
R⁴ and R⁶ are independently selected from the group consisting of O, S, Se, and BH₃.

n is 1, 2, or 3.

Preferred embodiments for R¹, R², R³, R⁴, R⁵, R⁶ are disclosed in WO 2011/015347 A1 and may be selected accordingly in the present invention.

For example, in an embodiment, the RNA of the present invention comprises a phosphorothioate-cap-analog. Phosphorothioate-cap-analogs are specific cap analogs in which one of the three non-bridging O atoms in the triphosphate chain is replaced with an S atom, i.e., one of R⁴, R⁵ or R⁶ in Formula (I) is S. Phosphorothioate-cap-analogs have been described by Kowalska et al., 2008, RNA, 14:1119-1131, as a solution to the undesired decapping process, and thus to increase the stability of RNA in vivo. In particular, the substitution of an oxygen atom for a sulphur atom at the beta-phosphate group of the 5′-cap results in stabilization against Dcp2. In that embodiment, which is preferred in the present invention, R⁵ in Formula (I) is S; and R⁴ and R⁶ are O.

In a further embodiment, the RNA of the present invention comprises a phosphorothioate-cap-analog wherein the phosphorothioate modification of the RNA 5′-cap is combined with an “anti-reverse cap analog” (ARCA) modification. Respective ARCA-phosphorothioate-cap-analogs are described in WO 2008/157688 A2, and they can all be used in the RNA of the present invention. In that embodiment, at least one of R² or R³ in Formula (I) is not OH, preferably one among R² and R³ is methoxy (OCH₃), and the other one among R² and R³ is preferably OH. In a preferred embodiment, an oxygen atom is substituted for a sulphur atom at the beta-phosphate group (so that R⁵ in Formula (I) is S; and R⁴ and R⁶ are O). It is believed that the phosphorothioate modification of the ARCA ensures that the α, β, and γ phosphorothioate groups are precisely positioned within the active sites of cap-binding proteins in both the translational and decapping machinery. At least some of these analogs are essentially resistant to pyrophosphatase Dcp1/Dcp2. Phosphorothioate-modified ARCAs were described to have a much higher affinity for elF4E than the corresponding ARCAs lacking a phosphorothioate group.

A respective cap analog that is particularly preferred in the present invention, i.e., m₂^{7,2-O}GppₛpG, is termed beta-S-ARCA (WO 2008/157688 A2; Kuhn et al., 2010, Gene Ther. 17:961-971). Thus, in one embodiment of the present invention, the RNA of the present invention is modified with beta-S-ARCA. beta-S-ARCA is represented by the following structure:

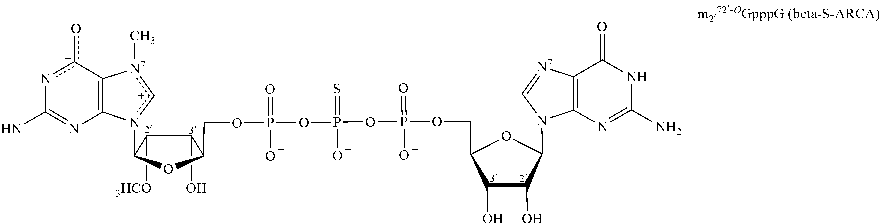

In general, the replacement of an oxygen atom for a sulphur atom at a bridging phosphate results in phosphorothioate diastereomers which are designated D1 and D2, based on their elution pattern in HPLC. Briefly, the D1 diastereomer of beta-S-ARCA″ or “beta-S-ARCA (D1)” is the diastereomer of beta-S-ARCA which elutes first on an HPLC column compared to the D2 diastereomer of beta-S-ARCA (beta-S-ARCA (D2)) and thus exhibits a shorter retention time. Determination of the stereochemical configuration by HPLC is described in WO 2011/015347 A1.

In a first particularly preferred embodiment of the present invention, RNA of the present invention is modified with the beta-S-ARCA (D2) diastereomer. The two diastereomers of beta-S-ARCA differ in sensitivity against nucleases. It has been shown that RNA carrying the D2 diastereomer of beta-S-ARCA is almost fully resistant against Dcp2 cleavage (only 6% cleavage compared to RNA which has been synthesized in presence of the unmodified ARCA 5′-cap), whereas RNA with the beta-S-ARCA (D1) 5′-cap exhibits an intermediary sensitivity to Dcp2 cleavage (71% cleavage). It has further been shown that the increased stability against Dcp2 cleavage correlates with increased protein expression in mammalian cells. In particular, it has been shown that RNAs carrying the beta-S-ARCA (D2) cap are more efficiently translated in mammalian cells than RNAs carrying the beta-S-ARCA (D1) cap. Therefore, in one embodiment of the present invention, RNA of the present invention is modified with a cap analog according to Formula (I), characterized by a stereochemical configuration at the P atom comprising the substituent R⁵ in Formula (I) that corresponds to that at the P_{β} atom of the D2 diastereomer of beta-S-ARCA. In that embodiment, R⁵ in Formula (I) is S; and R⁴ and R⁶ are O. Additionally, at least one of R² or R³ in Formula (I) is preferably not OH, preferably one among R² and R³ is methoxy (OCH3), and the other one among R² and R³ is preferably OH.

In a second particularly preferred embodiment, RNA of the present invention is modified with the beta-S-ARCA (D1) diastereomer. This embodiment is particularly suitable for transfer of capped RNA into immature antigen presenting cells, such as for vaccination purposes. It has been demonstrated that the beta-S-ARCA (D1) diastereomer, upon transfer of respectively capped RNA into immature antigen presenting cells, is particularly suitable for increasing the stability of the RNA, increasing translation efficiency of the RNA, prolonging translation of the RNA, increasing total protein expression of the RNA, and/or increasing the immune response against an antigen or antigen peptide encoded by said RNA (Kuhn et al., 2010, Gene Ther. 17:961-971). Therefore, in an alternative embodiment of the present invention, RNA of the present invention is modified with a cap analog according to Formula (I), characterized by a stereochemical configuration at the P atom comprising the substituent R⁵ in Formula (I) that corresponds to that at the P_{β} atom of the D1 diastereomer of beta-S-ARCA. Respective cap analogs and embodiments thereof are described in WO 2011/015347 A1 and Kuhn et al., 2010, Gene Ther. 17:961-971. Any cap analog described in WO 2011/015347 A1, wherein the stereochemical configuration at the P atom comprising the substituent R⁵ corresponds to that at the P_{β} atom of the D1 diastereomer of beta-S-ARCA, may be used in the present invention. Preferably, R⁵ in Formula (I) is S; and R⁴ and R⁶ are O. Additionally, at least one of R² or R³ in Formula (I) is preferably not OH, preferably one among R² and R³ is methoxy (OCH₃), and the other one among R² and R³ is preferably OH.

In one embodiment, RNA of the present invention is modified with a 5′-cap structure according to Formula (I) wherein any one phosphate group is replaced by a boranophosphate group or a phosphoroselenoate group. Such caps have increased stability both in vitro and in vivo. Optionally, the respective compound has a 2′-O- or 3′-O-alkyl group (wherein alkyl is preferably methyl); respective cap analogs are termed BH₃-ARCAs or Se-ARCAs. Compounds that are particularly suitable for capping of mRNA include the β-BH₃-ARCAs and β-Se-ARCAs, as described in WO 2009/149253 A2. For these compounds, a stereochemical configuration at the P atom comprising the substituent R⁵ in Formula (I) that corresponds to that at the P_{β} atom of the D1 diastereomer of beta-S-ARCA is preferred.

In one embodiment, the 5′ cap can be a trinucleotide AU(cap1) having the following structure:

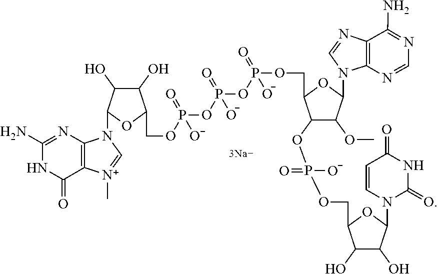 [SMILES: COC1C(OP(=O)([O-])OCC2OC(n3ccc(=O)[nH]c3=O)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In some embodiments, the capping analog may be selected from G[5′]ppp[5′]G, m⁷G[5′]ppp[5′]G, m₃^{2,2,7}G[5′]ppp[5′]G, m₂^{7,30}G[5]ppp[5] G (3′-ARCA), m₂^{7,2-O}GppG (2′-ARCA), m₂^{7,2-O}GppₛpG D1 (β-S-ARCA D1), m₂^{7,2-O}GppₛpG D2 (β-S-ARCA D2), m⁷ (3′OMeG) (5′)ppp(5′) (2′OMeA)pG (CleanCap® Reagent AG (3′ OMe), Catalog-No.: S2968), m⁷G (5′)ppp(5′) (2′OMeA)pG (CleanCap® Reagent AG, Catalog-No.: S2-668), CleanCap® Reagent AU-(N-7114), m⁷G (5′)ppp(5′) (2′OMeA)pU.

In some embodiments, in step (a) the population of RNA molecules may be contacted with an excess of catalytic nucleic acid molecules. For example, in step (a) the population of RNA molecules may be contacted with the catalytic nucleic acid molecules in a molar ratio of RNA molecules to catalytic nucleic acid molecules of about 1:1 to about 1:20, or about 1:1 to about 1:10. For example, a molar ratio of RNA molecules to catalytic nucleic acid molecules may be about 1:2.5, about 1:5 or about 1:10. Preferred is a ratio of about 1:2.5.

In some embodiments, the length of the 5′ terminal fragment allows discrimination between a capped 5′ terminal fragment and a non-capped 5′ terminal fragment. In some embodiments, the capped 5′ terminal fragment and a non-capped 5′ terminal fragment may differ by 1 to 3 nucleotides in length.

In some embodiments, the 5′ terminal fragment obtained in step (a) may have length of at least 5 nt, at least 10 nt, or at least 15 nt. In some embodiments, the 5′ terminal fragment obtained in step (a) may have length of up to 35 nt or up to 50 nt. In some embodiments, the lengths of the 5′ terminal fragment may be in the range of 5-50 nt, 10-50 nt or 15-30 nt.

In some embodiments, the 5′ terminal fragment preferably is subjected to step (b) in the reaction mixture comprising the reactants of the cleavage reaction of step (a).

In some embodiments, in step (a) the 5′ terminal fragment may be obtained in a mixture with the at least one 3′ fragment, the catalytic nucleic acid molecule and/or an uncleaved RNA molecule.

Cleavage in step (a) of the method as described herein may be essentially complete. In some embodiments, in step (a) the 5′ terminal fragment may be obtained in a mixture with the at least one 3′ fragment and the catalytic nucleic acid molecule.

Cleavage in step (a) of the method as described herein may be incomplete. In some embodiments, in step (a) the 5′ terminal fragment may be obtained in a mixture with the at least one 3′ fragment, the catalytic nucleic acid molecule, and an uncleaved RNA molecule.

In an embodiment, in step (b), the 5′ terminal fragments and the at least one 3′ fragment present in the population of cleaved RNA molecules obtained in step (a) are separated away or purified, at least partially, from each other. This separation (purification) step results in a population that is enriched for 5′ terminal fragments, i.e., the quantity of 5′ terminal fragments as a percentage of all cleaved and uncleaved RNA molecules present in the separated (or purified) enriched population obtained in step (b) is greater than the quantity of 5′ terminal fragments as a percentage of all cleaved and uncleaved RNA molecules present in the population obtained in step (a). In some embodiments, the enriched population of 5′ terminal fragments is in solution. In an embodiment, the enriched population of 5′ terminal fragments obtained in step (b), for example, in solution, can be used in the methods of the invention for analyzing the 5′ cap structure of an RNA molecule.

In some embodiments, the increase in percentage of the quantity of 5′ terminal fragments can be at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%. In some embodiments, the increase in percentage is in the range of 50% to 95%, 60% to 95%, 70% to 95%, 80% to 95%, 90% to 95%, or 95% to 99%. In some embodiments, the increase in percentage is 1.5-fold, 2-fold, 3-fold, 4-fold, 5-fold, 6-fold, 7-fold, 8-fold, 9-fold, 10-fold, 50-fold, 100-fold or higher.

In some embodiments, step (b) may comprise subjecting the mixture obtained in step (a) to chromatography using a silica-based stationary phase, under conditions allowing the at least partial separation of the 5′ terminal fragment from the at least one 3′ fragment, the uncleaved RNA molecule and/or the catalytic nucleic acid molecule.

Any silica-based material suitable for chromatography may used, for example in a column and/or on a membrane. Suitable silica-based materials are known to the skilled person.

In some embodiments, step (b) may comprise two separate chromatography steps, wherein in each step a silica-based stationary phase is used. Examples 1 and 2 describe typical protocols of silica-based chromatography with two separate columns.

In some embodiments, in the first chromatography step, the long RNAs, including the uncleaved, full-length RNA and the long 3′ fragments may remain on the solid phase, while the short 5′ terminal fragments were collected in the flow-through fraction. The mobile phase may be a mixture of the sample obtained in step (a), an alcohol and an aqueous buffer in a ratio allowing at least partial separation of the 5′ terminal fragment from the uncleaved RNA molecules and/or the at least one 3′ fragment.

In some embodiments, in the first chromatography step, the ratio of the alcohol and the aqueous buffer is smaller than 1, i.e. an excess of aqueous buffer in view of the alcohol is used. The volume ratio of the alcohol and the aqueous buffer may be in the range of about 2:3 to about 3:4, with an excess of aqueous buffer in view of the alcohol. In some embodiments, the mobile phase may contain the sample obtained in step (a) and the alcohol/buffer mixture in a volume ratio in the range of about 1:5 to about 1:7.

In some embodiments, in the second chromatography step, the 5′ terminal fragments may be bound to the solid phase. The mobile phase may be a mixture of the sample obtained in the first chromatography step, an alcohol, and an aqueous buffer, in a ratio allowing binding of the 5′ terminal fragment to the solid phase.

In some embodiments, in the second chromatography step, the ratio of the alcohol and the aqueous buffer is larger than 1, i.e. an excess of alcohol in view of the aqueous buffer is used. The volume ratio of the alcohol and the aqueous buffer may be in the range of about 5:1 to about 20:1, for example about 10:1. In some embodiments, the mobile phase may contain the sample obtained in the first chromatography step and the alcohol/buffer mixture in a volume ratio in the range of about 2:1 to about 1:1.

The 5′ fragments bound to the second column can be eluted with water. Suitable protocols are known to the skilled person.

In some embodiments, in the two chromatography steps, the alcohol may be independently a C₁₋₆ alcohol, preferably a C₁₋₃ alcohol, such a methanol, ethanol, 1-propanol or 2-propanol, or a mixture thereof. Ethanol is preferred.

The aqueous buffer may be any suitable buffer, known to the skilled person.

“Separating at least partially” or “at least partial separation” in step (b) of the method of the invention relates to complete or incomplete separation of the 5′ terminal fragment from the at least one 3′ fragment and/or any other component of the reaction mixture obtained in step (a).

In some embodiments, step (b) may also comprise subjecting the mixture obtained in step (a) to PAGE, under conditions allowing the at least partial separation of the 5′ terminal fragment from the at least one 3′ fragment and/or the uncleaved RNA molecule. PAGE may further comprise the steps of (i) isolating at least one band of interest from the PAGE gel, said at least one band comprising the 5′ terminal fragment, and (ii) eluting the 5′ terminal fragment from the isolated at least one band obtained in step (i).

In some embodiments, step (b) may also comprise contacting the mixture obtained in step (a) with oligo dT nucleotides under conditions allowing the at least partial separation of the 5′ terminal fragment from the at least one 3′ fragment and/or uncleaved RNA molecules. By the oligo dT nucleotides, an RNA molecule comprising a poly(A) sequence may be bound to a solid phase. mRNA molecules typically contain a poly(A) sequence. In some embodiments, the oligo dT nucleotides may be attached to plastic or magnetic beads or are attached to biotin. In some embodiments, the beads form a column.

According to the invention, in one embodiment, a poly(A) sequence comprises or essentially consists of or consists of at least 20, preferably at least 26, preferably at least 40, preferably at least 80, preferably at least 100 and preferably up to 500, preferably up to 400, preferably up to 300, preferably up to 200, and in particular up to 150, A nucleotides, and in particular about 120 A nucleotides. In this context “essentially consists of” means that most nucleotides in the poly(A) sequence, typically at least 50%, and preferably at least 75% by number of nucleotides in the “poly(A) sequence”, are A nucleotides (adenylate), but permits that remaining nucleotides are nucleotides other than A nucleotides, such as U nucleotides (uridylate), G nucleotides (guanylate), C nucleotides (cytidylate). In this context “consists of” means that all nucleotides in the poly(A) sequence, i.e. 100% by number of nucleotides in the poly(A) sequence, are A nucleotides. The term “A nucleotide” or “A” refers to adenylate.

In some embodiments, the catalytic nucleic acid molecule may be labeled, for example with biotin.

In some embodiments, the catalytic nucleic acid molecule may be attached to a surface. The surface may be a magnetic or plastic bead or particle.

In some embodiments, the separating step (b) may further comprise separating the 5′ terminal fragment from the catalytic nucleic acid molecule under conditions allowing the at least partial separation of the 5′ terminal fragment from the catalytic nucleic acid molecule. In some embodiments, the separating may comprise contacting the mixture of step (a) to a material that binds to the labeled catalytic nucleic acid molecule under conditions allowing the at least partial separation of the 5′ terminal fragment from the labeled catalytic nucleic acid molecule. The label may be biotin.

In some embodiments, steps (b) and (c) may be separate steps. As described herein, in step (b) the 5′ terminal fragment obtained in step (a) is separated at least partially from the at least one 3′ fragment, resulting in a population of 5′ terminal fragments. In some embodiments, in step (b) a capped 5′ terminal fragment is preferably not separated from a non-capped 5′ terminal fragment.

In some embodiments, in step (c), the amount of RNA molecules having the 5′ cap structure may be determined in the population of 5′ terminal fragments obtained in step (b). In some embodiments, in step (c), capped 5′ terminal fragments may be distinguished from uncapped 5′ fragments, for example by a chromatographic method. In some embodiments, the capped 5′ terminal fragment may one, two or three nucleotides longer than the non-capped 5′ terminal fragment.

In some embodiments, step (c) may comprise gel electrophoresis, spectroscopic analysis, mass spectrometry, liquid chromatography and/or sequencing. In some embodiments, gel electrophoresis may be PAGE. In some embodiments, mass spectrometry may be LC-MS. In some embodiments, liquid chromatography may be HPLC or UPLC.

In some embodiments, in steps (b) and (c), different methodologies may be used. For example, in step (b), chromatography using a silica-based stationary phase, an oligo dT based separation, elution from the PAGE, and/or a separation method using immobilized catalytic nucleic acids may be used, whereas in step (c), PAGE, mass spectrometry, HPLC and/or UPLC may be used.

In some embodiments, step (c) may comprise determination of the amounts of the capped 5′ terminal fragment and the non-capped 5′ terminal fragment. By the amounts of the capped 5′ terminal fragment and the non-capped 5′ terminal fragment, the capping efficiency may be determined.

In some embodiments, the percentage of capped 5′ terminal fragments may be calculated relative to the total amount of 5′ terminal fragments. The percentage may be calculated as mole % or weight %.

In some embodiments, the method of the invention may further comprise step (d): analyzing the cap structure in the capped 5′ terminal fragments. An analysis of the cap structure may comprise the analysis of bonding between the capping analog and the RNA molecule. A capping analog may comprise two 3′ positions (termed herein “first” and “second” 3′ positions) which both may be capable to bind to the 5′ end of the RNA molecule. In the first position the ribose may connected to guanosine (G), and in the second position the ribose may be connected to m⁷G. The term “orientation” or “correct orientation” describes a bonding of the RNA molecule to the first 3′ position, resulting in efficient translation of mRNA, while “reverse orientation” describes a bonding of the RNA molecule to the second 3′ position, resulting in reduced translation efficiency. Analysis of the cap structure may comprise determination of the amount of correctly orientated cap structure and/or percentage of correctly orientated cap structure with respect to the total amount of capped molecules. For example, treatment of a 5′ terminal fragment capped in the correct orientation with pyrophosphatase results in the cleavage of m7G from the 5′terminal fragment, and treatment of a 5′ terminal fragment capped in the reverse orientation with pyrophosphatase results in the cleavage of G from the 5′terminal fragment, allowing discrimination of the correct orientation and the reverse orientation.

Yet another aspect of the present invention is a method for determining capping efficiency in a population of RNA molecules, said method comprising the steps:
(a) contacting a catalytic nucleic acid molecule with a population of RNA molecules, which population comprises one or more RNA molecules comprising a cleavage site for the catalytic nucleic acid molecule and a 5′ cap structure under conditions allowing the cleavage of the RNA molecules to produce a 5′ terminal fragment and at least one 3′ fragment,
(b) separating the 5′ terminal fragment obtained in step (a) at least partially from the at least one 3′ fragment, resulting in a population of 5′ terminal fragments, and
(c) determining in the population of 5′ terminal fragments obtained in step (b) the amount of RNA molecules having the 5′ cap structure.

Embodiments of steps (a), (b) and (c) are described herein in the context of the method analyzing a population of RNA molecules of the invention.

Yet another aspect of the present invention is a method for analyzing an RNA molecule, comprising the steps:
(i) synthesizing an RNA molecule,
(ii) capping the RNA synthesized in (i), and
(iii) analyzing the RNA molecule by the method for analyzing a population of RNA molecules, of the invention, as described herein.

In step (i), any known RNA synthesis method may be used. In some embodiments, the RNA molecule may be synthesized by in-vitro transcription and/or solid-phase synthesis, as described herein in the context of the method for analyzing a population of RNA molecules of the invention.

In some embodiments, in step (ii), the RNA synthesized in step (i) may be capped by enzymatic or/and co-transcriptional capping, as described herein in the context of the method for analyzing a population of RNA molecules of the invention.

Yet another aspect of the present invention is a method for capped RNA synthesis quality control, comprising the steps:
(i) synthesizing an RNA molecule,
(ii) capping the RNA synthesized in (i) and
(iii) analyzing the RNA molecule by the method for analyzing a population of RNA molecules of the invention, as described herein.

In step (i), any known RNA synthesis method may be used. In some embodiments, the RNA molecule may be synthesized by in-vitro transcription and/or solid-phase synthesis, as described herein in the context of the method for analyzing a population of RNA molecules of the invention.

In some embodiments, in step (ii), the RNA synthesized in step (i) may be capped by enzymatic or/and co-transcriptional capping, as described herein in the context of the method for analyzing a population of RNA molecules of the invention.

In an embodiment, the separating and determining steps are not performed simultaneously using a PAGE gel or HPLC.

Yet another aspect of the present invention is a catalytic nucleic acid molecule, comprising
(i) a sequence selected from SEQ ID NO: 1-25,
(ii) a sequence having at least 80% identity with any one of SEQ ID NO: 1-25, and/or
(iii) a fragment of (i) and/or (ii),
   wherein the catalytic nucleic acid molecule comprises a catalytic core.

SEQ ID NO: 1-25 are described herein in the context of the method for analyzing a population of RNA molecules of the invention.

In some embodiments, the catalytic nucleic acid molecule described in this aspect of the invention may be an isolated catalytic nucleic acid molecule. An “isolated molecule” as used herein, is intended to refer to a molecule which is substantially free of other molecules such as other cellular material. The term “isolated nucleic acid” means according to the invention that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant techniques.

The term “recombinant” in the context of the present invention means “made through genetic engineering”. Preferably, a “recombinant object” such as a recombinant cell in the context of the present invention is not occurring naturally.

In some embodiments, the catalytic nucleic acid molecule of the invention may be an RNA molecule, in particular a ribozyme.

Yet another aspect of the present invention is a nucleic acid molecule, comprising
(i) a sequence of SEQ ID NO: 26 or SEQ ID NO:27,
(ii) a sequence having at least 90% identity, preferably at least 95%, 96%, 97%, 98%, or 99% identity with SEQ ID NO: 26 and/or SEQ ID NO:27.

In some embodiments, the nucleic acid molecule described in this aspect of the invention may be an isolated nucleic acid molecule.

In some embodiments, the nucleic acid molecule of this aspect may be an RNA molecule.

In some embodiments, the nucleic acid molecule of this aspect may comprise a catalytic core of a ribozyme.

In some embodiments, the nucleic acid molecule of this aspect may be a ribozyme.

In some embodiments, the nucleic acid molecule of this aspect may comprise at least one modified nucleotide, as described herein.

Yet another aspect of the present invention is the use of the catalytic nucleic acid molecule, as described herein, in a method of the invention for analyzing a population of RNA molecules, in a method of the invention for determining capping efficiency in a population of RNA molecules, in a method of the invention for analyzing an RNA molecule, and/or a method of the invention for capped RNA synthesis quality control, as described herein.

The present invention is described in detail and is illustrated by the figures and examples, which are used only for illustration purposes and are not meant to be limiting. Owing to the description and the examples, further embodiments which are likewise included in the invention are accessible to the skilled worker.

### DESCRIPTION OF THE DRAWINGS

FIG. 1: Ribozyme-mediated cleavage to quantify capping efficiency of in vitro-transcribed mRNA. Ribozyme (Rz) anneals to IVT mRNA and cleaves the 5′-end of the IVT mRNA at 37° C. in the presence of Mg⁺⁺. Substrate cleavage results in a mixture of RNAs: short capped and uncapped 5′ cleavage products (5′CPs), long 3′ cleavage products (3′CPs), long uncleaved RNAs, and the Rz. The mixture is purified using a process with two silica-based columns, whereby the long RNAs and 3′CPs are depleted on the first column membrane by using specific salt and ethanol conditions. The collected flow-through containing the short capped and uncapped 5′CPs is applied to the second column, bound on its membrane, and eluted in water. The purified 5′CPs and Rz are visualized using 21% PAGE, 8 M urea or analyzed with liquid chromatography and mass spectrometry (LC-MS), allowing quantification of capping efficiency of the IVT mRNA.

FIG. 2: Optimization of molar ratio of ribozyme to IVT mRNA substrate. A fixed amount of U-containing or m¹ψ-containing uncapped mRNA was cleaved using increasing amounts of Rz1, and the resulting mixture was visualized using 21% PAGE, 8 M urea. The cleavage efficiency of Rz1 was assessed for increasing Rz to IVT mRNA substrate molar ratios, based on ratios between uncleaved RNA (112 nt long) and 3′ cleavage products (3′CP=90 nt long). Molar ratios of Rz to RNA substrate from 1 to 10 were tested, resulting in approximately 50 to 70% cleavage. Rz was used as a control (ctrl). 5′CP, 5′ cleavage products; nt, nucleotides.

FIG. 3: Ribozyme-mediated cleavage effectively assesses capping efficiencies of IVT mRNAs by visualization and quantification using denaturing polyacrylamide gel electrophoresis. Ribozyme-(Rz) mediated cleavage (using Rz1, Rz2, and Rz5) of U-containing or m1ψ′-containing RNAs: uncapped (−), enzymatic cap0 (E0), enzymatic cap1 (E1), and ARCA (A0) were either purified on silica-based columns and then visualized using 21% PAGE, 8 M urea (purified, upper panel) or visualized using 21% PAGE, 8 M urea without silica-based column purification (unpurified, lower panel). Rz was used as a control on both the upper and lower panels while uncleaved mRNA was in addition used as a control (ctrl) on the lower panels. 5′CP, 5′ cleavage products (upper: capped, lower: uncapped); nt, nucleotides. Capping efficiencies (%) of the IVT mRNAs visualized here are shown in Table 5.

FIG. 4: Ribozyme-mediated cleavage effectively assesses capping efficiencies of IVT mRNAs of different lengths. The image shows 21% PAGE, 8 M urea visualization of Rz5-cleaved and silica-column purified TEV, m1ψ-containing, beta-S-ARCA (D1) or CleanCap® Reagent AG (3′ OMe), cap1 (CC1) capped IVT mRNAs. The IVT mRNAs ranged from 1.1 kb to 9.4 kb in length. Rz, ribozyme; 5′CP, 5′ cleavage product.

FIG. 5: Ribozyme-mediated cleavage assay detects increase in capping efficiency after enzymatic capping of co-transcriptionally capped IVT mRNA. GCG transcription start site (TSS), hAg, U-containing IVT mRNAs, which were co-transcriptionally D1 capped or D1+enzymatically capped (D1+E1), were Rz1 cleaved, silica-column purified, and visualized using 21% PAGE, 8 M urea. Rz, ribozyme; 5′CP, 5′ cleavage product.

FIG. 6: Ribozyme-mediated cleavage assay is superior to RNase H cleavage assay. RNase H probe (P1) was hybridized, and RNase H cleaved a set of U- or m¹⁴-containing RNAs: uncapped (−), enzymatic cap0 (E0), enzymatic cap1 (E1), and ARCA (A0). Cleaved RNA fragment mixtures were either applied to silica-based columns for purification and visualized using 21% PAGE, 8 M urea (purified) or visualized using 21% PAGE, 8 M urea without silica-based column purification (unpurified). White arrows: additional+1 nt band, dashed square: RNA degradation caused by RNase H. RNase H probe P1 or uncleaved RNA (ctrl) were used as controls. 5′CP, 5′ cleavage products; nt, nucleotides.

FIG. 7: LC-MS analysis of ribozyme-mediated cleavage products for quantification and characterization of capped products from Rz1 cleaved and silica-column purified enzymatically capped and 2′-O-methylated (E1), human alpha globin (hAg), m1ψ-containing erythropoietin (EPO) mRNA. The enzymatical capping procedure gives 7MeGpppA (OMe) GGCGAACU*AGU*AU*U*-CU*U*CU*GGU*Cp (MW=8,334) and 7MeGpppAGGCGAACU*AGU*AU*U*CU*U*CU*GGU*Cp (MW=8,319) in a 7:2 ratio; resulting from incomplete 2′O-methyl transfer. (A) UPLC and (B) MS profiles. Rz, ribozyme; Cap, capped product; Capm, capped methylated product; Capu, capped unmethylated product; Cap+G, minor product; AU, arbitrary units.

FIG. 8: LC-MS analysis of ribozyme-mediated cleavage products for quantification and characterization of capped products from Rz1 cleaved and silica-column purified CleanCap® Reagent AG (3′ OMe)=cap1 (CC1), human alpha globin (hAg), m¹⁴-containing erythropoietin (EPO) mRNA. Expected capped product (MW=8,347) is detected in >99%. (A) UPLC and (B) MS profiles. Rz, ribozyme; Cap, capped product; AU, arbitrary units.

FIG. 9: Ribozyme-mediated cleavage assay using ribozymes targeting NCH type sites (Rz6, Rz7, Rz8, Rz9 and Rz10; SEQ ID NOS: 6 to 10) showing efficacious cleavage and releasing capped and uncapped short 5′ cleavage products.

### EXAMPLE 1

In this Example, ribozymes (Rz) were designed to specifically cleave IVT mRNA at a unique position in close proximity to the 5′-end, releasing capped or uncapped short 5′ cleavage products in a range of 10-30 nt. The well-defined 5′ cleavage products cut off by the ribozyme from the capped mRNA differ by one nucleoside in length, specifically the cap structure itself, compared to the cleavage products cut from an uncapped RNA.

These products were purified using silica-based columns and visualized/quantified them using denaturing polyacrylamide gel electrophoresis (PAGE) or liquid chromatography and mass spectrometry (LC-MS). Using this technology, the capping efficiencies of IVT mRNAs with different features was determined, which include: different cap structures, diverse 5′ untranslated regions, different nucleoside-modifications, and diverse lengths. Taken together, the ribozyme cleavage assays we developed are fast and reliable for the analysis of capping efficiency for R&D purposes and as a general quality control for mRNA-based therapeutics.

RNAs from the cleavage reaction can be analyzed directly in denaturing polyacrylamide gel electrophoresis (PAGE). The present example demonstrates that purifying the RNAs from the cleavage reaction using a silica-based column and loading the resulting cleaned, short 5′ cleavage product RNAs onto the gel results in better reproducibility and improves visualization and quantification.

The RNAs are electrophoresed under conditions in which the difference of the 5′ cleavage products is detectable. The stained and visualized 5′ cleavage products released from the capped or uncapped mRNAs are quantified via their gel band intensity, and capping efficiency is assessed. The short purified 5′ cleavage products were also analyzed using LC-MS, allowing additional characterization, such as determination of methylation status or minor capped products.

### 1. Materials and Methods

### 1.1. Templates for In Vitro Transcription

Templates for in vitro transcription were generated by linearizing plasmids containing different coding sequences flanked by sequences corresponding to the 5′ untranslated region (UTR) of human alpha globin (hAg) or 5′-leader of tobacco etch virus (TEV), a constant 3′UTR, and 100 nt-long poly(A) tail [15, 16]. The linearization was performed with the restriction enzymes Earl or Bbsl (both from New England Biolabs).

### 1.2. In Vitro Transcription and Capping of RNA

RNA ranging in size from 100 nt to 9.4 kb was synthesized using the MEGAscript T7 transcription kit (Thermo Fisher Scientific). The reaction included UTP for generating standard IVT mRNA or N1-methylpseudouridine 5′-triphosphate (m1ψ′TP) (TriLink) for nucleoside-modified mRNA, in which 100% of uridine were substituted with m1ψ′. In a subset of RNAs, the sequences of the first 3 transcribed nucleotides were GCG, GGA, AGC, AGG, or AGA. Cap analog, ARCA-G (TriLink, N-7003), beta-S-ARCA (D1, BioNTech SE) [17], or CleanCap® Reagent AG (3′ OMe) (TriLink, N-7413) was added to the transcription reaction to generate mRNA with cap0 (A0), cap0 (D1), or cap1 (CC1), respectively. Vaccinia virus capping enzymes (New England Biolabs) were used according to the manufacturer's instructions to enzymatically cap the synthesized mRNA and generate RNA with cap0 (E0) or cap1 (E1). RNA quality was tested using 1.4% agarose gel electrophoresis [18], and RNA concentration was measured using a NanoDrop spectrophotometer (Thermo Scientific).

1.3. Design of ribozymes and RNase H probe

Five hammerhead ribozymes were designed for this study (Table 3).

TABLE 3
Characteristics of the designed ribozymes.

| 5′UTR | Rz | Rz sequence (5′-3′) |
|---|---|---|
| hAG | Rz1 | UGU GGG CUG AUG AGG CCG UGA GGC CGA AAC |
| | | CAG AAG AAU |
| hAG | Rz2 | GGG GAC CAG AAG AAC UGA UGA GGC CGU GAG |
| | | GCC GAA ACUm AmGmUm UmCmGm |
| hAG | Rz3 | UGU GGG CUG AUG AGG CCG UGA GGC CGA AAC |
| | | CAG AA |
| hAG | Rz4 | AGU CUG UGG GCU GAU GAG GCC GUG AGG CCG |
| | | AAA CCA GAA GAA |
| TEV | Rz5 | GUA UAC UGA UGA GGC CGU GAG GCC GAA |
| | | IUmUm GmUmGm UmUmGm AmGmAm CmUmAm |
| | | GmUmUm UmAm |

Hammerhead Rz catalytic core sequences are underlined;
hAg, human alpha globin;
I, inosine;
m, 2′-O-Met;
TEV, tobacco etch virus;
Rz, ribozyme;
5′UTR, 5′ untranslated region.

Rz1, Rz2, Rz3, and Rz4 cleave the hAg 5′UTR after GUC, GUA, GUC, and GUC triplets, respectively [19, 20]. Rz3 differs from Rz1 by exhibiting a 4 nt shorter 3′ arm, while Rz4 was designed with a 4 nt longer 5′ arm and 1 nt shorter 3′ arm. Rz5 was engineered to contain inosine (I) and cleave the TEV 5′UTR after an ACA triplet [21]. To increase stability of the annealed Rz: target complex, 2′-O-methylated nucleotides (Nm) were incorporated into the last 7 or 19 nucleotide positions of Rz2 and Rz5, respectively. The RNase H probe (5′-dGdAdC dCdAdG AmAmGm AmAmUm AmCmUm Am-3′), in which deoxynucleotides (dNs) were incorporated, was designed according to Beverly et al. [13]. Ribozymes and RNase H probe were synthesized by Metabion and their quality was confirmed by electrospray ionization time-of-flight mass spectrometry (ESI-TOF).

1.4. mRNA Cleavage by Ribozyme

Ribozyme cleavage reactions contained 0.2 to 0.6 μM mRNA, and a 2.5-fold molar excess of ribozyme over mRNA substrate, in 10 mM Tris and 10 mM MgCl2. Firstly, ribozyme was annealed to mRNA in the reaction mix without MgCl2 by incubating first at 95° C. for 2 min and then at room temperature for 5 min. The cleavage reactions were started by adding MgCl2 and performed at 37° C. for 1 h, then processed further or frozen at −20° C. A 2.5-fold molar excess of ribozyme over mRNA was found to be optimal after testing a range from 1.0- to 10.0-fold molar excess of ribozyme over mRNA substrate.

1.5. mRNA Cleavage by RNase H

The RNase H cleavage assay was done by annealing 5-fold molar excess of RNase H probe with mRNA substrate by incubating at 92° C. for 2 min, then stepwise cooling down (65° C. for 2 min, 55° C. for 2 min and 40° C. for 2 min) in a buffer containing 50 mM Tris and 100 mM NaCl. After annealing, 125 UM RNase H (New England Biolabs) and 10 mM MgCl2 were added to the reaction mix, followed by incubation at 37° C. for 1 h. Reactions were further processed or frozen at −20° C.

### 1.6. Purification of Cleaved, Short RNA Fragments Using Silica-Based Columns

The cleaved, short RNA fragments—from a mixture of the cleaved or uncleaved long RNA fragments present in the ribozyme or RNase H-mediated cleavage reaction—were purified by adapting a procedure for RNA separation using the RNeasy Mini Kit (Qiagen). First, 100 μl cleavage reaction mixture was mixed with 350 μl RLT buffer (lysis buffer from the RNeasy Mini Kit) and 250 μl 100% ethanol and applied to the column. The long RNAs, including the uncut, full-length mRNA and the long 3′ cleaved fragments remained on the column while the short 5′ cleavage products were collected in the flow-through fraction. Next, 50 μl RLT buffer and 500 μl 100% ethanol were added to the collected flow-through fraction (700 μl), and the mix was applied to the second silica column sequentially in two aliquots of 625 μl with intervening centrifugation at 9,600×g for 15 sec on a Heraeus Fresco 17 Centrifuge (Thermo Scientific), with the temperature kept at 22° C. to 23° C. throughout all centrifugation steps. Under these conditions, the short 5′ cleavage products and ribozyme bound to the column were washed with 500 μl RPE buffer (wash buffer from the RNeasy Mini Kit) and centrifuged at 9,600×g for 15 sec, followed by a wash step with 500 μl 100% ethanol and centrifugation at 9,600×g for 2 min. To remove the remaining ethanol, the column was transferred to a clean collection tube and centrifuged at 17,000×g for 1 min. The column was then transferred to a clean 1.5 ml tube, and the short RNA fragments were eluted by adding 30 μl RNase-free water to the column followed by centrifugation at 13,800×g for 1 min. Typically, 20 μg RNA was purified per column for RNAs <5 kb long or 60 μg for RNAs >5 kb long.

1.7. Purification of Cleaved, Short Fragments by Elution from Polyacrylamide Gel

As a second option, purification of cleaved, short fragments was performed according to a method modified from Nilsen 2013 [22], whereby separation was followed by elution from a 21% polyacrylamide gel prepared with 8 M urea. Samples were separated in 2 mini gels processed parallel on a Bio-Rad Mini-Protean Tetra Cell in Tris-borate-EDTA (TBE) buffer. The first gel was stained with SYBR® Gold nucleic acid gel stain (Thermo Fisher Scientific S11494) diluted 1:10,000 in TBE buffer and used as a reference, while the corresponding bands of interest were cut from the second, unstained gel run in parallel. The excised gel fragments were transferred into 400 μl elution buffer (20 mM Tris-HCl, 3 M sodium acetate, 1 mM EDTA, 0.25% SDS), frozen for 15 min on dry ice and stored overnight at room temperature to allow release of RNA into the solution. After centrifugation at 17 000×g for 10 min, the supernatant contained RNA, which was extracted with an equal volume of acid-phenol/chloroform, followed by chloroform. Isopropanol precipitation was done, and recovered RNA was dissolved in 10 μl RNase-free water.

### 1.8. Visualization and Analysis of Cleaved, Short Fragments by PAGE

RNA fragments cleaved by ribozyme or RNase H were diluted 1:1 with Gel Loading Buffer II (Ambion): 1) directly after cleavage, 2) after cleavage and silica-column purification, or 3) after purification from a gel as described above. For optimal loading, the reaction volumes were constant between the samples and the concentrations of purified short RNA fragments were between 10-70 ng/μl. RNA samples were denatured at 65° C. for 10 min, then separated using 21% PAGE, 8 M urea, in TBE buffer for 2-2.5 h, stained with SYBR® Gold and visualized by ultraviolet light using a Gel Doc EZ system (Bio-Rad). The image was analyzed using Volume tools in Image Lab 5.0 software (Bio-Rad). The images of RNA bands were selected which corresponded to 1) the slower moving capped 5′ cleavage product RNA, 2) the 1 nucleotide-shorter, faster moving uncapped 5′ cleavage product RNA, and 3) background of the same area. To quantify capping efficiency, the relative intensities of bands corresponding to the capped and uncapped RNA were measured for each RNA sample and background values were subtracted. The total intensity of the 2 bands combined was considered 100%. The calculated value for the band corresponding to the capped RNA represents the capping efficiency.

### 1.9. LC-MS Analysis of Ribozyme-Cleaved Products

LC-MS was performed using an Acquity Ultra Performance Liquid Chromatography (UPLC) system (Waters) coupled to a Xevo TQ-S mass spectrometer (Waters) equipped with an electrospray source operating in negative ionization mode. MS spectra were acquired over a range from m/z 400 to 2,000. All samples were chromatographed on an Acquity Beh C18 column (Waters, 2.1×50 mm; 1.7 μm particle size) at 60° C. column temperature. The analytes were separated in a gradient of 16.6 mM triethylamine (TEA; VWR), 100 mM hexafluoroisopropanol (HFIP; Sigma) and 10% methanol, Ultra LC-MS grade (Carl Roth) as buffer A and 16.6 mM TEA, 100 mM HFIP and 95% methanol as buffer B, with a flow rate of 0.3 ml/min. The gradients applied for oligonucleotides >20 nt length were: 0% for 1.5 min, 0 to 7% over 3.5 min, 7 to 15% over 6.25 min, 15 to 40% buffer B over 4.5 min.

### 2. Results

2.1. Ribozyme Assays to Quantify Capping Efficiency of IVT mRNA

Human alpha globin (hAg) and TEV 5′ untranslated regions (5′UTRs) are among the most widely used 5′UTRs for therapeutic IVT mRNAs [16, 23, 24]. Ribozymes were designed that targeted the 5′UTR sequences of hAg or TEV (Table 4, see methods section). All five of the ribozymes designed here were expected to form the well-described hammerhead structure and cleave the targeted RNA after defined nucleotide triplets [19, 25]. Ribozymes cleave most efficiently after the AUC or GUC triplets, while other triplets can be also targeted but with the following declining cleavage efficiency: GUA, AUA, CUC >AUU, UUC, UUA >GUU, CUA >UUU, CUU [21]. Rz1, Rz3, and Rz4 were designed to cleave after GUC and Rz2 after GUA. Rz5 contained inosine (I) which allows recognition and cleavage after the ACA triplet in the TEV 5′UTR [21].

TABLE 4
Length of the designed
ribozymes and example cleavage products.

| | | | Example of | | |
|---|---|---|---|---|---|
| | | Rz | uncapped 5′CP | 5′ CP (nt) | |
| 5′UTR | Rz | (nt) | (capped: +7MeG) | −cap | +cap |
| hAG | Rz1 | 39 | pppGCGAACUAGUAUUCUUCUG | 22 | 23 |
| | | | GUC>CCCACAGACU . . . | | |
| hAG | Rz2 | 45 | pppGCGAACUAGUA>UUCUUCU | 11 | 12 |
| | | | GGUCCCCACAGACU . . . | | |
| hAG | Rz3 | 35 | pppGCGAACUAGUAUUCUUCUG | 22 | 23 |
| | | | GUC>CCCACAGACU . . . | | |
| hAG | Rz4 | 42 | pppGCGAACUAGUAUUCUUCUG | 22 | 23 |
| | | | GUC>CCCACAGACU . . . | | |
| TEV | Rz5 | 47 | pppGGAAUAAACUAGUCUCAAC | 25 | 26 |
| | | | ACAACA>UAUACAAA . . . | | |

Recognition sequences are underlined;
cleavage positions are indicated as N>;
CP, cleavage product;
hAg, human alpha globin;
TEV, tobacco etch virus;
Rz, ribozyme;
5′UTR, 5′ untranslated region.

Each of the ribozyme-mediated cleavage reactions produced short capped and uncapped RNA 5′ cleavage products that differed from each other solely in their lengths-due to the cap structure, the capped RNA was exactly one nucleotide longer. The ribozymes were designed to cleave off 10-30 nt-long products from the target RNA, while the ribozymes were 35-47 nt, allowing separation and differentiation of the 5′ cleavage products and the ribozyme (Table 4). The Rz2 and Rz5 sequences that are complementary to the targeted RNA also contained 2′-O-methylated nucleotides to enhance cleavage by increasing stabilities of the formed double-strand structures.

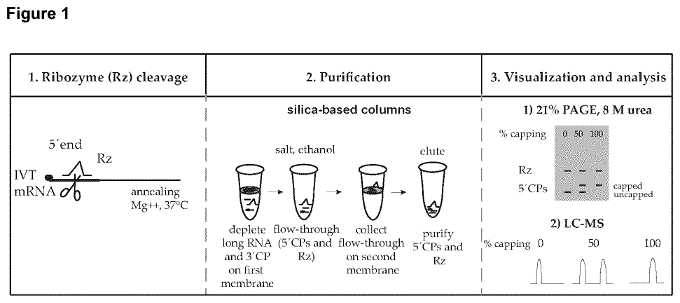

To quantify capping efficiency, Rz was annealed to the mRNA substrate (FIG. 1). In the presence of Mg⁺⁺ ions, the 5′-end of the IVT mRNA was cleaved, resulting in a mixture of: short capped and/or uncapped 5′ cleavage products, long 3′ cleavage products, long uncleaved RNA, and the Rz. Optimized conditions were used to purify short Rz-cleaved fragments using silica-based MinElute Qiagen columns, prior to visualization using 21% PAGE, 8 M urea or LC-MS analysis (FIG. 1). In this case, 5′ cleavage products together with the ribozyme are purified and further visualized/quantified. Purification of the 5′ cleavage products is necessary for LC-MS analysis and also improves RNA visualization using 21% PAGE, 8 M urea.

An alternative purification approach was implemented on the PAGE-separated samples, by extracting and eluting the 5′ cleavage products from the gel (see methods section, data not shown). This process results in the elimination of both the uncut and 3′-end cleaved RNA products as well as the ribozyme from the mixture, which may be an advantage in the case where LC-MS analysis is planned using Rz and 5′ cleavage products that overlap. However, this purification step is experimentally time consuming and not easily scalable. Using this method, it is possible to purify 2-4 samples in parallel in 2 days, while using silica-based columns allows the purification of 12 samples in parallel in less than 1 day with the option to scale up.

Here, we describe an assay to assess capping efficiency. The method consists of a ribozyme cleavage reaction, purification of cleaved fragments, and visualization of capped and uncapped products using 21% PAGE, 8 M urea or LC-MS analysis.

### 2.2. Ribozyme Cleavage Assay Optimization

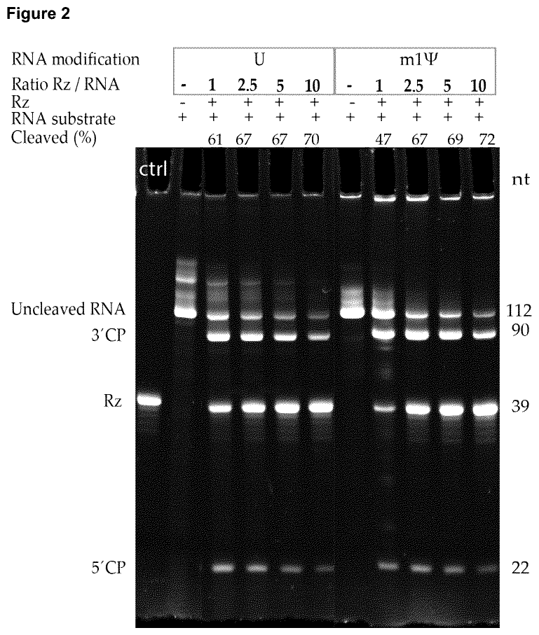

To identify the optimal molar ratio of the Rz to RNA substrate for the cleavage reaction, a 112 nt-long U- or m1ψ-containing mRNA substrate was selected. Using the aforementioned short substrates allowed the detection and differentiation of the uncleaved RNA, 5′ and 3′ cleavage products, and the Rz separated on the same gel. FIG. 2 shows the 112 nt-long uncleaved RNA, the 22 and 90 nt-long 5′ and 3′ cleavage products, and the 39 nt-long Rz1 that were detected using 21% PAGE, 8 M urea. Increasing molarities of Rz over the RNA substrate were tested for both U- and m1ψ-containing RNAs. A 2.5-fold excess molarity of Rz over the RNA substrate was selected and used in all subsequent experiments.

For further optimization, various temperature settings during the cleavage reaction were tested. The reaction was performed with Rz1, Rz3, and Rz4 at 25° C., 37° C., and 50° C. as detailed in section 1.4.

Temperature did not have any notable effect on Rz cleavage in this experiment, and the same capping efficiency results were obtained using different temperature settings. However, reactions performed at 50° C. led to more prominent degradation (data not shown). Takagi et al. and Sawata et al. found that below 25° C. the product dissociation step became the rate-determining and at the temperature of 37° C. no burst kinetics were detected and ribozyme chemical cleavage was the rate-determining step [26, 27]. Taking their and our findings into consideration, cleavage reactions at 37° C. were used in subsequent experiments.

2.3. Capping Efficiency Quantification after Visualizing 5′ Cleavage Products Using 21% PAGE, 8 M urea

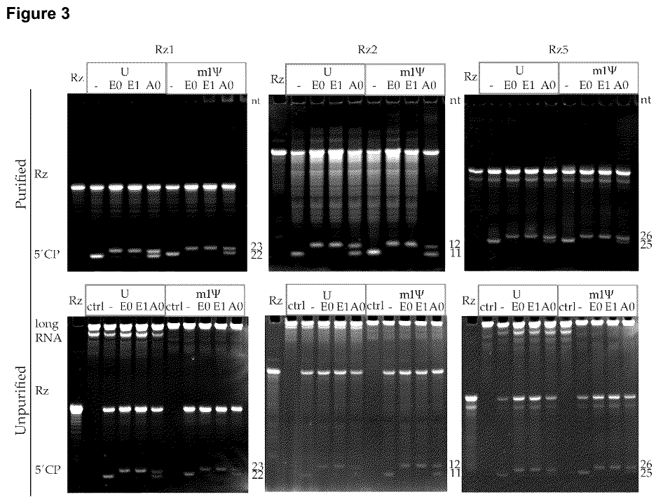

To measure capping efficiency, short capped and uncapped 5′ cleavage products were visualized using 21% PAGE, 8 M urea. As a proof-of-principle experiment, cleavage reactions were done using erythropoietin (EPO)-encoding mRNA. GCG transcription start site (TSS) and hAg 5′UTR-containing IVT mRNAs were cleaved using Rz1 and Rz2. EPO-encoding GGA TSS and TEV 5′UTR-containing IVT mRNAs were cleaved using Rz5. The following U- and m1ψ′-containing RNAs were tested: uncapped (−), enzymatically capped without 2′-O-methylation (E0), enzymatically capped and 2′-O-methylated (E1), or ARCA co-transcriptionally capped (A0). Using 21% PAGE, 8 M urea, bands representing ribozyme (Rz) and short capped and uncapped 5′ cleavage products were detected at the expected sizes (FIG. 3).

3′end cleavage products or uncleaved long RNAs were observed in 21% PAGE, 8 M urea gels after ribozyme-mediated cleavage reactions without a silica-based column purification step. Depletion of long RNA fragments and an increase in signals representing the short capped and uncapped 5′ cleavage products were observed after purification using silica-based columns. As expected, visualization using 21% PAGE, 8 M urea showed the presence of bands at the bottom of the gel in all uncapped control (−) RNA samples. Furthermore, in all enzymatically capped (E0 and E1) samples, a capped high-intensity band was observed in the majority of cases, while uncapped bands were either not observed or had lower intensities, indicating a high capping efficiency of enzymatically capped RNAs. In contrast, both capped and uncapped bands were detected in comparable intensities in ARCA (A0) samples, irrespective of the RNA or Rz used.

Approximately 84-100% capping efficiencies were detected for all 12 enzymatically capped (E0 and E1) samples independent of the 5′-end and ribozyme used, while ARCA (A0) samples showed 34-53% capping efficiencies for hAg 5′UTR and 67-77% for TEV 5′UTR-containing RNAs (Table 5). When the % capping efficiencies of purified E0 RNA and corresponding 2′-O-methylated E1 RNA were compared, in 5 of 6 cases+/−1% differences were seen, showing the high reproducibility of the assay.

TABLE 5
Capping efficiencies of IVT mRNAs quantified after ribozyme-mediated
cleavage assays and visualization using 21% PAGE, 8M urea.

| 5′UTR | | hAg | | | | TEV | |
|---|---|---|---|---|---|---|---|
| Purification | | Unpurified | | Purified | | Unpurified | Purified |
| Assay | | Rz1 | Rz2 | Rz1 | Rz2 | Rz5 | |
| Cap | Modification | Capping efficiency (%) | | | | | |
| E0 | U | 100 | 86 | 90 | 95 | 93 | 89 |
| | m1Ψ | 88 | 100 | 95 | 96 | 100 | 99 |
| E1 | U | 100 | 84 | 91 | 95 | 95 | 90 |
| | m1Ψ | 92 | 100 | 95 | 96 | 98 | 95 |
| A0 | U | 52 | 51 | 52 | 52 | 75 | 67 |
| | m1Ψ | 40 | 48 | 53 | 34 | 77 | 69 |

Enzymatically capped without 2′-O-methylation (E0), enzymatically capped and 2′-O-methylated (E1), ARCA co-transcriptionally capped (A0), unmodified/uridine-containing (U), m1Ψ-containing (m1Ψ), hAg, human alpha globin; TEV, tobacco etch virus.

Capping efficiencies of silica-column purified RNAs were consistently less variable when compared to ribozyme-cleaved RNAs that were not silica-column purified. In 10 of 12 cases using different batches of purified RNAs, both E0 and E1 showed capping efficiencies between 90 and 96%, while for the same RNAs that were not silica-column purified, the observed capping efficiency range was 88 to 100% (Table 5). Therefore, purification using silica columns is recommended as a standard part of the procedure, not only for LC-MS analysis but also for visualization using 21% PAGE, 8 M urea and capping efficiency quantification.

2.4. Capping Assay by Ribozyme-Mediated Cleavage Effectively Assesses Capping Efficiencies of Diversely Capped IVT mRNAs of Different Lengths

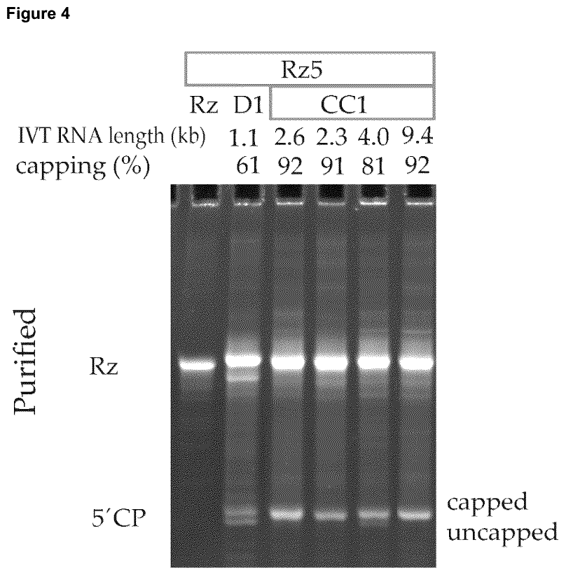

To test if ribozyme-mediated cleavage can detect capping of IVT mRNA with different lengths, a Rz5 cleavage reaction was performed (without modifying the method described above) on five IVT mRNAs ranging from 1.1 kb to 9.4 kb in length. The 1.1-kb long beta-S-ARCA (D1) capped TEV 5′UTR, m1ψ-containing RNA showed 61% capping efficiency while the 2.3-9.4 kb long CleanCap® Reagent AG (3′ OMe), cap1 (CC1) RNAs showed 81-92% capping efficiency (FIG. 4). There was no correlation observed between the % capping and RNA length. The method described here successfully assessed capping efficiencies of IVT mRNAs of different lengths capped using diverse cap structures.

2.5. Ribozyme-Mediated Cleavage Assay Detects Increase in Capping Efficiency after Additional Enzymatic Capping of Co-Transcriptionally Capped RNA

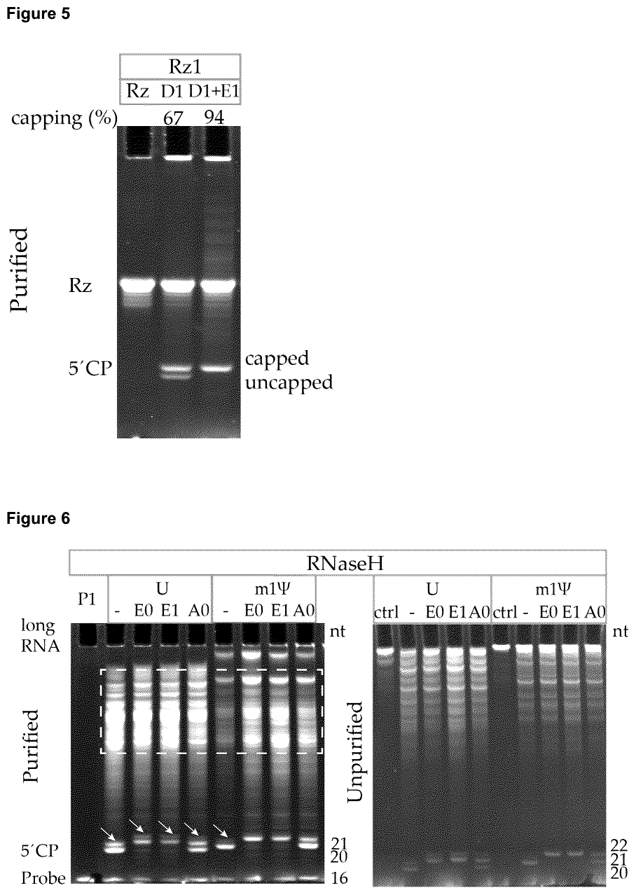

To further test the ribozyme-mediated cleavage assay performance, the co-transcriptionally capped D1 U-containing GCG TSS hAg 5′UTR IVT mRNA was subsequently subjected to enzymatic capping (E1). While co-transcriptionally D1 capped IVT mRNA initially showed 67% capping efficiency, after subsequent E1 capping, capping efficiency increased to 94% (FIG. 5). This finding confirms that a significant portion of 5′ cleavage products in D1 are indeed uncapped and do not represent T7 RNA polymerase potentially skipping the first G in the transcription start which might result in the same RNA fragment that is 1 nt shorter than the capped fragment and equal to the size of the uncapped fragment.

### 2.6. Ribozyme-Mediated Cleavage Assay Performance Superior to RNase H Cleavage Assay

To compare the ribozyme-mediated and RNase H-mediated cleavage assays, we developed six RNase H probes that could anneal to the hAg 5′UTR sequence. RNase H cleavage reactions were performed as described in the methods section, and the probes were screened (data not shown). A probe (P1) containing a stretch of six DNA nucleotides (dNs) and ten 2′-O-methylated RNA nucleotides was selected due to superior performance (Table 6).

TABLE 6
RNase H cleavage assay probe and products.

| | | | | 1) 5′CP | | |
|---|---|---|---|---|---|---|
| | | | | sequence | | |
| | | | | w/o cap | | |
| | | Probe | Probe | 2) minor | 5′CP | |
| | Probe | sequence | size | cleavage | size (nt) | |
| 5′UTR | name | (5′-3′) | (nt) | product | −cap | +cap |
| hAg | Probe 1 | GACCAGA- | 16 | 1) GCGAA | 20 | 21 |
| | (P1) | mAmGmAmA- | | CUAGUAUU | | |
| | | mUmAmCmU | | CUUCUGG | | |
| | | mAm | | 2) GCGAA | 21 | 22 |
| | | | | CUAGUAUU | | |
| | | | | CUUCUGGU | | |

DNA nucleotides in bold, dN;
m, 2′-O-Met;
hAg, human alpha globin.

The 5′-ends of the hAg GCG-starting enzymatically capped (E0 or E1) or co-transcriptionally ARCA capped (A0) U- and m¹⁴-containing RNAs were RNase H cleaved (FIG. 6). RNase H cleavage confirmed high capping efficiencies for enzymatic RNAs previously detected by ribozyme-mediated cleavage (purified hAg GCG: 75-85%). For ARCA samples, rather low capping efficiencies of 37-47% were obtained (FIG. 6, Table 7) which is in accordance with the data shown in section 2.3.

In contrast to ribozyme-mediated cleavage, RNase H cleavage resulted in an additional band. The band was 1 nt longer (21 or 22 nt long) and appeared in all U-containing samples including uncapped

RNA, next to or overlapping the expected short RNA fragments of 20 nt and 21 nt, corresponding to the uncapped and capped enzymatic 5′ cleavage products, respectively (FIG. 6). This finding indicates that RNase H cleaved at two positions: at the expected position after the fourth DNA nucleotide and with lower efficiency at the position after the fifth DNA nucleotide, thereby complicating capping efficiency analysis in U-containing samples (FIG. 6). In addition, in all RNase H-cleaved samples a large spread of nonspecific long RNA fragments not present in the no-enzyme control was observed (FIG. 6). These nonspecific long RNA fragments of various sizes could not be depleted by silica-based column purification. Their presence made capping efficiency quantification from the 21% PAGE, 8 M urea gels less reproducible, and this may lead to complex LC-MS analysis.

Accordingly, RNase H cleavage using specific probes can be used to quantify capping efficiency using 21% PAGE, 8 M urea. However, ribozyme-mediated cleavage in contrast to RNase H cleavage leads to single-position cleavage and does not result in a smear of nonspecific long fragments, allowing reliable quantification using 21% PAGE, 8 M urea or LC-MS analysis.

TABLE 7
Quantification of capping efficiencies of IVT mRNAs after RNase
H cleavage assay and visualization using 21% PAGE, 8 M urea.

| | |
|---|---|
| 5′UTR | hAg |

| | | |
|---|---|---|
| Purification | Unpurified | Purified |

| | | |
|---|---|---|
| Cap | Modification | Capping efficiency (%) |

| | | | |
|---|---|---|---|
| E0 | U | 75 | 82 |
| | m1Ψ | 89 | 85 |
| E1 | U | 82 | 78 |
| | m1Ψ | 82 | 83 |
| A0 | U | 56 | 47 |

Enzymatically capped without 2′-O-methylation (E0), enzymatically capped and 2′-O-methylated (E1), ARCA co-transcriptionally capped (A0), unmodified/uridine-containing (U), m¹⁴-containing (m¹⁴′); hAg, human alpha globin.

2.7. LC-MS analysis of capping efficiency using ribozyme-mediated cleavage assay

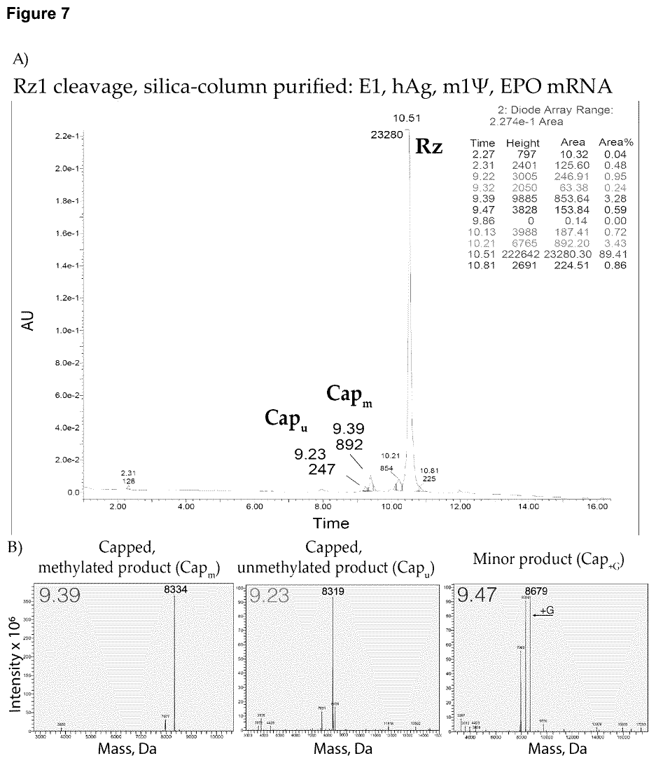

As a proof of principle, LC-MS analysis was done on ribozyme-cleaved and silica-based column purified short 5′ cleavage products from enzymatically capped and 2′-O-methylated (E1) or CleanCap® Reagent AG (3′ OMe) (CC1) AGG TSS, hAg 5′UTR, m1ψ-containing IVT mRNAs. UPLC and MS profiles of E1 capped IVT mRNA (FIG. 7) showed:

68% of the expected major product
(7MeGpppA(Ome)GGCGAACU*AGU*AU*U*CU*U*CU*GGU*C>p),
20% unmethylated product
(7MeGpppAGGCGAACU*AGU*AU*U*CU*U*CU*GGU*C>p),
and 1% additional product (+G).

The detection of 89% of the E1 capped product (for AGG TSS IVT mRNA) is in agreement with the detected 95-96% for E1 capped RNAs using 21% PAGE, 8 M urea; furthermore, a different batch and GCG TSS RNA was used for PAGE.

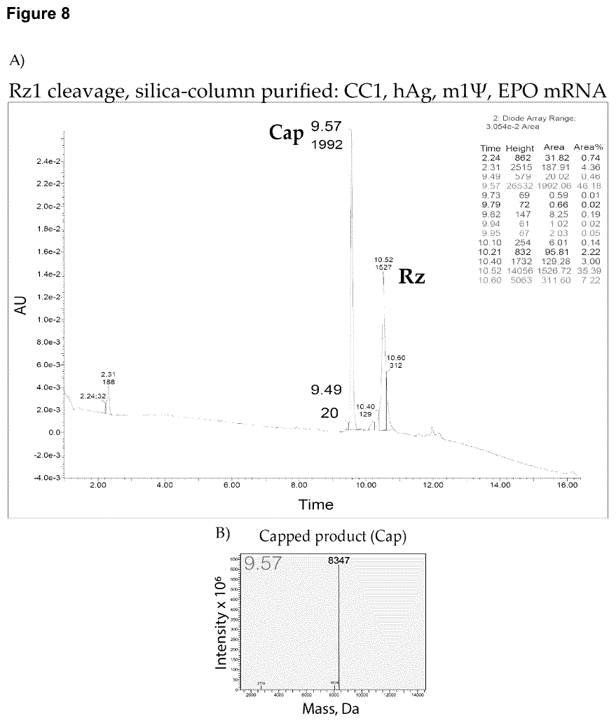

UPLC and MS profiles of CleanCap® Reagent AG (3′ OMe), N-7413 TriLink (CC1) EPO mRNA (FIG. 8) showed >99% capping, which is also in agreement with >90% capping efficiency obtained by screening of >100 CC1-capped IVT mRNAs using 21% PAGE, 8 M urea (FIG. 4 and data not shown). Therefore, the ribozyme-mediated cleavage assay combined with silica-based column purification is highly compatible with LC-MS analysis. As shown here, application to LC-MS creates additional possibilities for characterizing 5′ cleavage products, such as determining methylation status or distinguishing minor capped products.

### 3. Discussion

Quality control of mRNA vaccines and therapeutics is necessary at every stage of development, from preclinical studies to clinical applications, as well as to support marketing authorizations. The cap structure on the mRNA molecule determines mRNA translation and thus correlates with the therapeutic efficacy of the mRNA [7-9]. In the process of mRNA production, capping of the mRNA can be performed enzymatically or co-transcriptionally. Both strategies result in a mixture of capped and uncapped mRNA molecules. In this study, we developed an assay to detect the capping efficiency based on a ribozyme-mediated cleavage reaction. After this reaction, the next step is silica-column purification and visualization and quantification using 21% PAGE, 8 M urea or LC-MS. Visualization using 21% PAGE, 8 M urea allows the analysis of a large number of mRNA samples in parallel without the need for specific equipment. In addition, the method described here is compatible with LC-MS, allowing in-depth characterization, where not only the percentage of capping, but methylation status and potential minor capped byproducts can be detected from the same sample.

As previously discussed, the current approaches to assess capping efficiency have limitations, such as the necessity for radioactive labeling [10-12] or a rather insensitive detection of capping levels [14]. Beverly et al. developed an assay to assess capping efficiency based on RNase H cleavage of specific biotin-tagged probes followed by purification using streptavidin-coated magnetic beads and LC-MS analysis [13]. Here, we directly compared our developed ribozyme-mediated cleavage assay that is targeted to the hAg 5′UTR with the RNase H cleavage assay we designed for the same 5′UTR. As expected, ribozyme cleaved only at one position in the IVT mRNA, and after silica-based purification only three short RNA fragments (the ribozyme as well as the capped and uncapped 5′ cleavage products) were observed on the gel, allowing precise quantification. In contrast, RNase H cleaved at two positions, resulting in capped and uncapped 5′ cleavage products and an additional unexpected band 1 nt longer that also appeared in the uncapped RNA control sample. This made quantification using the RNase H assay cumbersome. These results are in accordance with the observations of Beverly et al., who also reported two cleavage sites after RNase H cleavage and biotin-tagged analysis [13]. Moreover, we observed that RNase H cleavage also yields a large number of nonspecific long cleaved RNA fragments, which are expected to negatively influence quantification. We conclude that the assay based on ribozyme cleavage developed in this study shows major benefits compared to other assays for capping detection.

Design of ribozyme for use in these assays in order to quantify the capping efficiency depends on the 5′terminal sequence of the target mRNA. Thus, a ribozyme cleavage site is required in a structurally accessible region between position 10 and 30 nt from the 5′ end of mRNA. In this study hammerhead ribozymes were designed to cleave after GUC (Rz1, Rz3, Rz4), GUA (Rz2) or ACA (Rz5) triplets. In addition to such triplets, other NUH triplets (H represents A, C or U) which can be targeted by hammerhead ribozymes can be also employed (NUH cleavage efficiency in decreasing order is: AUC>AUA, CUC>AUU, UUC, UUA>GUU, CUA>UUU, CUU [21]). Incorporation of inosine in the ribozyme recognition sequence (as in Rz5), allows additional targeting at NCH triplets (e.g. ACA). The possibility for targeting both canonical NUH triplets and non-canonical NCH sites, significantly improves the versatility of this assay by allowing selection for the most accessible cleavage site within the 5′UTR [21]. The assays developed and optimized here could be used to reproducibly quantify the capping efficiencies of U- or m1ψ-containing IVT mRNAs having diverse caps, containing different 5′UTRs, and of different lengths. The 89 to 100% capping efficiencies observed after using a vaccinia virus capping enzyme system confirm previous findings of 88 to 98% capping reported by Beverly et al. [13]. Moreover, we show that diverse cap structures can lead to diverse capping efficiencies that are relatively consistent between different IVT mRNA batches produced using the same cap structure. For example, while enzymatic capping and co-transcriptionally capped CleanCap® Reagent AG (3′ OMe) (TriLink) typically yielded >90% capping efficiencies, co-transcriptional capping using ARCA-G (TriLink) or beta-S-ARCA (D1) led to lower capping efficiencies ranging from 34 to 77%.

Taken together, the ribozyme-mediated cleavage assays developed in this study are useful assays for facile, fast, and reliable analysis of capping efficiency for research and development purposes or as a quality control for hAg and TEV 5′UTR-containing mRNA-based therapeutics. The same methods for ribozyme assay design, short fragment purification, and visualization or quantification by gel electrophoresis or LC-MS may be used to develop ribozyme assays targeted against other 5′UTRs, allowing wider applicability in the mRNA therapeutics field.

### EXAMPLE 2

Two-Step Silica-Based Column Chromatography of Catalytic Nucleic-Acid (e.g. Ribozyme) Cleaved IVT mRNA.
Mix the catalytic nucleic-acid (e.g. ribozyme) cleaved IVT mRNA mixture with aqueous buffer and then add ethanol (buffer volume > or =ethanol volume) and apply to the first silica-based column.
After centrifugation step, the long RNAs, including the uncut, full-length mRNA and the long 3′ cleaved fragments remained on the column while the short 5′ cleavage products were collected in the flow-through fraction.
Next, buffer and ethanol were added to the collected flow-through fraction (aqueous buffer <ethanol volume), and the mix was applied to the second silica column.
Under these conditions, the short 5′ cleavage products bound to the column were washed and centrifuged in multiple steps until eluted from the column.
Purification of 5′end short products improve reproducibility of UPLC and/or LC-MS analysis and improves visualization and quantification from polyacrylamide gel.

### EXAMPLE 3

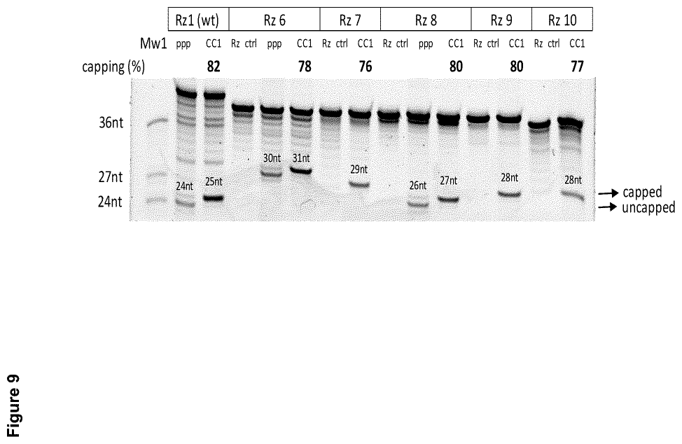

In a similar experimental protocol as that set forth above in Example 1, this example compares the ribozyme used in Example 1 (Rz1) with five other ribozymes, each of which target NCH cleavage sites, e.g., ACA, CCA, CCC. In particular, ribozymes Rz6 to Rz10 (SEQ ID Nos: 6-10) and Rz1 were used to cleave capped (CC1) and uncapped (ppp) RNA molecules. The results presented in FIG. 9 show efficacious cleavage and releasing of capped or uncapped short 5′ cleavage products in a range of 10-31 nucleotides using ribozymes targeting NCH type sites in the RNA molecule.

### REFERENCES

1. Sahin U, Karikó K, Türeci Ö. mRNA-based therapeutics—developing a new class of drugs. Nat Rev Drug Discov. 2014; 13:759-80. doi: 10.1038/nrd4278.
2. Sahin U, Derhovanessian E, Miller M, Kloke B, Simon P, Lower M, et al. Personalized RNA mutanome vaccines mobilize poly-specific therapeutic immunity against cancer. Nature. 2017; 547:222-6. doi: 10.1038/nature23003.
3. Pardi N, Hogan M J, Porter F W, Weissman D. mRNA vaccines—a new era in vaccinology. Nat Rev Drug Discov. 2018; 17:261-79. doi: 10.1038/nrd.2017.243.
4. Polack F P, Thomas S J, Kitchin N, Absalon J, Gurtman A, Lockhart S, et al. Safety and Efficacy of the BNT162b2 mRNA Covid-19 Vaccine. N Engl J Med. 2020; 383:2603-15. doi: 10.1056/NEJMoa2034577.
5. Anderson E J, Rouphael N G, Widge A T, Jackson L A, Roberts P C, Makhene M, et al. Safety and Immunogenicity of SARS-COV-2 mRNA-1273 Vaccine in Older Adults. N Engl J Med. 2020; 383:2427-38. doi: 10.1056/NEJMoa2028436.
6. Vlatkovic I. Non-Immunotherapy Application of LNP-mRNA: Maximizing Efficacy and Safety. Biomedicines 2021. doi: 10.3390/biomedicines9050530.
7. Ramanathan A, Robb G B, Chan S. mRNA capping: biological functions and applications. Nucleic Acids Res. 2016; 44:7511-26. doi: 10.1093/nar/gkw551.
8. Shimotohno K, Kodama Y, Hashimoto J, Miura Kl. Importance of 5′-terminal blocking structure to stabilize mRNA in eukaryotic protein synthesis. Proc Natl Acad Sci USA. 1977; 74:2734-8.
9. Paterson B M, Rosenberg M. Efficient translation of prokaryotic mRNAs in a eukaryotic cell-free system requires addition of a cap structure. Nature. 1979; 279:692-6.
10. Stepinski J, Waddell C, Stolarski R, Darzynkiewicz E, Rhoads R E. Synthesis and properties of mRNAs containing the novel “anti-reverse” cap analogs 7-methyl (3′-O-methyl) GpppG and 7-methyl (3′-deoxy) GpppG. RNA. 2001; 7:1486-95.
11. Grudzien E, Stepinski J, Jankowska-Anyszka M, Stolarski R, Darzynkiewicz E, Rhoads R E. Novel cap analogs for in vitro synthesis of mRNAs with high translational efficiency. RNA. 2004; 10:1479-87. doi: 10.1261/rna.7380904.
12. Anderson B R, Muramatsu H, Nallagatla S R, Bevilacqua P C, Sansing L H, Weissman D, Kariko K. Incorporation of pseudouridine into mRNA enhances translation by diminishing PKR activation. Nucleic Acids Res. 2010; 38:5884-92. doi: 10.1093/nar/gkq347.
13. Beverly M, Dell A, Parmar P, Houghton L. Label-free analysis of mRNA capping efficiency using RNase H probes and LC-MS. Anal Bioanal Chem. 2016; 408:5021-30. doi: 10.1007/s00216-016-9605-x.
14. Moya-Ramírez I, Bouton C, Kontoravdi C, Polizzi K. High resolution biosensor to test the capping level and integrity of mRNAs. Nucleic Acids Res. 2020; 48: e129. doi: 10.1093/nar/gkaa955.
15. Orlandini von Niessen, Alexandra G., Poleganov M A, Rechner C, Plaschke A, Kranz L M, Fesser S, et al. Improving mRNA-Based Therapeutic Gene Delivery by Expression-Augmenting 3′ UTRs Identified by Cellular Library Screening. Mol Ther. 2019; 27:824-36. doi: 10.1016/j.ymthe.2018.12.011.
16. Kariko K, Muramatsu H, Welsh F A, Ludwig J, Kato H, Akira S, Weissman D. Incorporation of pseudouridine into mRNA yields superior nonimmunogenic vector with increased translational capacity and biological stability. Mol Ther. 2008; 16:1833-40. doi: 10.1038/mt.2008.200.
17. Kowalska J, Lewdorowicz M, Zuberek J, Bojarska E, Wojcik J, Cohen L S, et al. Synthesis and properties of mRNA cap analogs containing phosphorothioate moiety in 5′,5′-triphosphate chain. Nucleosides Nucleotides Nucleic Acids. 2005; 24:595-600. doi: 10.1081/ncn-200061915.
18. Masek T, Vopalensky V, Suchomelova P, Pospisek M. Denaturing RNA electrophoresis in TAE agarose gels. Anal Biochem. 2005; 336:46-50. doi: 10.1016/j.ab.2004.09.010.
19. Karikó K, Megyeri K, Xiao Q, Barnathan E S. Lipofectin-aided cell delivery of ribozyme targeted to human urokinase receptor mRNA. FEBS Lett. 1994; 352:41-4.
20. Li F, Barnathan E S, Kariko K. Rapid method for screening and cloning cDNAs generated in differential mRNA display: Application of northern blot for affinity capturing of cDNAs. Nucleic Acids Res. 1994; 22:1764-5.
21. Ludwig J, Blaschke M, Sproat B S. Extending the cleavage rules for the hammerhead ribozyme: Mutating adenosine15.1 to inosine15.1 changes the cleavage site specificity from N16.2U16.1H17 to N16.2C16.1H17. Nucleic Acids Res. 1998; 26:2279-85.
22. Nilsen T W. Gel purification of RNA. Cold Spring Harb Protoc. 2013; 2013:180-3. doi: 10.1101/pdb.prot072942.
23. Kariko K, Kuo A, Barnathan E. Overexpression of urokinase receptor in mammalian cells following administration of the in vitro transcribed encoding mRNA. Gene Ther. 1999; 6:1092-100. doi: 10.1038/sj.gt.3300930.
24. Holtkamp S, Kreiter S, Selmi A, Simon P, Koslowski M, Huber C, et al. Modification of antigen-encoding RNA increases stability, translational efficacy, and T-cell stimulatory capacity of dendritic cells. Blood. 2006; 108:4009-17. doi: 10.1182/blood-2006-04-015024.
25. Scott W G, Horan L H, Martick M. The hammerhead ribozyme: structure, catalysis, and gene regulation. Prog Mol Biol Transl Sci. 2013; 120:1-23. doi: 10.1016/B978-O-12-381286-5.00001-9.
26. Takagi Y, Taira K. Temperature-dependent change in the rate-determining step in a reaction catalyzed by a hammerhead ribozyme. FEBS Lett. 1995; 361:273-6. doi: 10.1016/0014-5793 (95) 00192-c.
27. Sawata S, Shimayama T, Komiyama M, Kumar P K, Nishikawa S, Taira K. Enhancement of the cleavage rates of DNA-armed hammerhead ribozymes by various divalent metal ions. Nucleic Acids Res. 1993; 21:5656-60. doi: 10.1093/nar/21.24.5656.

1. A method for analyzing a population of RNA molecules, said method comprising the steps
(a) contacting a catalytic nucleic acid molecule with a population of RNA molecules, which population comprises one or more RNA molecules comprising a cleavage site for the catalytic nucleic acid molecule and a 5′ cap structure, under conditions allowing the cleavage of the RNA molecules to produce a 5′ terminal fragment and at least one 3′ fragment,
(b) separating the 5′ terminal fragment obtained in step (a) at least partially from the at least one 3′ fragment, resulting in a population of 5′ terminal fragments, and
(c) determining in the population of 5′ terminal fragments obtained in step (b) the amount of RNA molecules having the 5′ cap structure.

2. The method of claim 1, wherein the population of RNA molecules is a population of mRNA molecules, self-replicating RNA, ncRNA and/or sRNA.

3. The method of claim 1 or 2, wherein in step (a) the catalytic nucleic acid molecule is contacted with a population of RNA molecules obtained by in-vitro transcription or solid-phase synthesis.

4. The method of any one of the preceding claims, wherein in the RNA molecule, the cleavage site is located at least 5 nt downstream the 5′ end of the RNA molecule.

5. The method of any one of the preceding claims, wherein in the RNA molecule, the cleavage site is located at most 50 nt downstream the 5′ end of the RNA molecule.

6. The method of any one of the preceding claims, wherein the RNA molecule comprises a 5′ UTR.

7. The method of claim 6, wherein the 5′ UTR is selected form Human alpha globin (hAg) 5′ UTR and TEV 5′ UTR.

8. The method of any one of the preceding claims, wherein the RNA molecule comprises at least one cleavage site for the catalytic nucleic acid molecule.

9. The method of any one of the preceding claims, wherein the catalytic nucleic acid molecule cleaves at a cleavage site in a 5′ UTR sequence.

10. The method of any one of the preceding claims, wherein the quantity of 5′ terminal fragments as a percentage of all cleaved and uncleaved RNA molecules present in the obtained after step (b) is greater than the quantity of 5′ terminal fragments as a percentage of all cleaved and uncleaved RNA molecules present in the population prior to step (b), providing for an enriched or at least partially purified population of 5′ terminal fragments.

11. The method of any one of the preceding claims, wherein the catalytic nucleic acid molecule cleaves at a 5′-NUH-3′ cleavage site in the RNA molecule to produce a 5′ fragment comprising a NUH>p 3′end, wherein
N is selected from G, A, C and U; and
H is selected from A, C and U.

12. The method of any one of the preceding claims, wherein the catalytic nucleic acid molecule cleaves at a 5′-NCH-3′ cleavage site in the RNA molecule to produce a 5′ fragment comprising a NCH>p 3′ end, wherein
N is selected from G, A, C and U; and
H is selected from A, C and U.

13. The method of any one of the preceding claims, wherein the catalytic nucleic acid molecule is a ribozyme or a DNAzyme.

14. The method of any one of the preceding claims, wherein the catalytic nucleic acid molecule is a hammerhead ribozyme, a hairpin ribozyme, or a HDV ribozyme.

15. The method of any one of the preceding claims, wherein the catalytic nucleic acid molecule and/or the RNA molecule comprises at least one modified nucleotide.

16. The method of any one of the preceding claims, wherein the catalytic nucleic acid molecule comprises
(i) a sequence selected from SEQ ID NO: 1-25,
(ii) a sequence having at least 80% identity with any one of SEQ ID NO: 1-25, and/or
(iii) a fragment of (i) and/or (ii),
wherein the catalytic nucleic acid molecule comprises a catalytic core.

17. The method of claim 16, wherein
Am is independently selected from A and 2′-O-methyladenosine,
Gm is independently selected from G and 2′-O-methylguanosine,
Um is independently selected from U and 2′-O-methyluridine, and/or
Cm is independently selected from C and 2′-O-methylcytidine.

18. The method of any one of the preceding claims, wherein in step (a) the catalytic nucleic acid molecule is contacted with a population of RNA molecules capped by enzymatic or/and co-transcriptional capping in the presence of a capping analog.

19. The method of claim 18, wherein the capping analog is selected from G[5′]ppp[5′]G, m⁷ G[5′]ppp[5′]G, m₃^{2,2,7}G[5′]ppp[5′]G, m₂^{7,3′-O}G[5′]ppp[5′] G (3′-ARCA), m₂^{7,2′-O}GpppG (2′- ARCA), m₂^{7,2-O}GppₛpG D1 (β-S-ARCA D1), m₂^{7,2-O}GppₛpG D2 (β-S-ARCA D2), m⁷ (3′OMeG) (5′)ppp(5′) (2′OMeA)pG (CleanCap® Reagent AG (3′ OMe)) and m⁷G (5′)ppp(5′) (2′OMeA)pG (CleanCap® Reagent AG).

20. The method of any one of the preceding claims, wherein in step (a) the population of RNA molecules is contacted with an excess of catalytic nucleic acid molecules.

21. The method of any one of the preceding claims, wherein in step (a) the population of RNA molecules is contacted with the catalytic nucleic acid molecules in a molar ratio of RNA molecules to catalytic nucleic acid molecules of about 1:1 to about 1:20.

22. The method of any one of the preceding claims, wherein the length of the 5′ terminal fragment allows discrimination between a capped 5′ terminal fragment and a non-capped 5′ terminal fragment.

23. The method of claim 22, wherein the capped 5′ terminal fragment and a non-capped 5′ terminal fragment differ by 1 to 3 nucleotides in length.

24. The method of any one of the preceding claims, wherein the 5′ terminal fragment obtained in step (a) has a length of at least 5 nt.

25. The method of any one of the preceding claims, wherein the 5′ terminal fragment obtained in step (a) has a length of up to 50 nt.

26. The method of any one of the preceding claims, wherein in step (a) the 5′ terminal fragment is obtained in a mixture with the at least one 3′ fragment, the catalytic nucleic acid molecule and/or an uncleaved RNA molecule.

27. The method of claim 26, wherein step (b) comprises subjecting the mixture obtained in step (a) to chromatography using a silica-based stationary phase, under conditions allowing the at least partial separation of the 5′ terminal fragment from the at least one 3′ fragment, the uncleaved RNA molecule and/or the catalytic nucleic acid molecule.

28. The method of claim 27, comprising two separate chromatography steps, wherein a silica-based stationary phase is used.

29. The method of claim 26, wherein step (b) comprises subjecting the mixture obtained in step (a) to PAGE, under conditions allowing the at least partial separation of the 5′ terminal fragment from the at least one 3′ fragment and/or the uncleaved RNA molecule.

30. The method of claim 29, further comprising (i) isolating at least one band of interest from the PAGE gel, said at least one band comprising the 5′ terminal fragment, and (ii) eluting the 5′ terminal fragment from the isolated at least one band obtained in step (i).

31. The method of claim 26, wherein step (b) comprises contacting the mixture obtained in step (a) with oligo dT nucleotides under conditions allowing the at least partial separation of the 5′ terminal fragment from the at least one 3′ fragment and/or uncleaved RNA molecules.

32. The method of claim 31, wherein the oligo dT nucleotides are attached to plastic or magnetic beads or are attached to biotin.

33. The method of claim 32, wherein the beads form a column.

34. The method of any one of the preceding claims, wherein the catalytic nucleic acid molecule is labeled.

35. The method of claim 34, wherein the label is biotin.

36. The method of any one of the preceding claims, wherein the catalytic nucleic acid molecule is attached to a surface.

37. The method of claim 36, wherein the surface is a magnetic or plastic bead or particle.

38. The method according to any of the preceding claims wherein the separating step (b) further comprises separating the 5′ terminal fragment from the catalytic nucleic acid molecule under conditions allowing the at least partial separation of the 5′ terminal fragment from the catalytic nucleic acid molecule.

39. The method according to claim 38 wherein the separating comprises contacting the mixture of step (a) to a material that binds to the labeled catalytic nucleic acid molecule under conditions allowing the at least partial separation of the 5′ terminal fragment from the labeled catalytic nucleic acid molecule.

40. The method of any one of the preceding claims, wherein in step (b) the capped 5′ terminal fragment is not separated from a non-capped 5′ terminal fragment.

41. The method of any one of the preceding claims, wherein steps (b) and (c) are separate steps.

42. The method of any one of the preceding claims, wherein the capped 5′ terminal fragment is one, two or three nucleotides longer than the non-capped 5′ terminal fragment.

43. The method of any one of the preceding claims, wherein step (c) comprises gel electrophoresis, spectroscopic analysis, mass spectrometry, liquid chromatography and/or sequencing.

44. The method of claim 43, wherein gel electrophoresis is PAGE.

45. The method of any one of the preceding claims, wherein the amount of RNA molecules having the 5′ cap structure is determined in the at least partially purified 5′ terminal fragment obtained in step (b).

46. The method of any one of the preceding claims, wherein in step (c), the amounts of the capped 5′ terminal fragment and the non-capped 5′ terminal fragment are determined.

47. The method of any one of the preceding claims, wherein in step (c), the percentage of capped 5′ terminal fragments is calculated relative to the total amount of 5′ terminal fragments.

48. The method of any one of the preceding claims, further comprising
(d) analyzing the cap structure in the capped 5′ terminal fragments.

49. A method for determining capping efficiency in a population of RNA molecules, said method comprising the steps:
(a) contacting a catalytic nucleic acid molecule with a population of RNA molecules, which population comprises one or more RNA molecules comprising a cleavage site for the catalytic nucleic acid molecule and a 5′ cap structure, under conditions allowing the cleavage of the RNA molecules to produce a 5′ terminal fragment and at least one 3′ fragment,
(b) separating the 5′ terminal fragment obtained in step (a) at least partially from the at least one 3′ fragment, resulting in a population of 5′ terminal fragments, and
(c) determining in the population of 5′ terminal fragments obtained in step (b) the amount of RNA molecules having the 5′ cap structure.

50. A method for analyzing an RNA molecule, comprising the steps:
(i) synthesizing an RNA molecule,
(ii) capping the RNA synthesized in (i), and
(iii) analyzing the RNA molecule by the method of any one of the claims 1-55.

51. A method for capped RNA synthesis quality control, comprising the steps:
(i) synthesizing an RNA molecule,
(ii) capping the RNA synthesized in (i), and
(iii) analyzing the RNA molecule by the method of any one of claims 1-55.

52. The method of claims 50 and 51, wherein the RNA molecule is by in-vitro transcription and/or solid-phase synthesis.

53. The method of any one of the claims 50-52, wherein the RNA molecule is capped by enzymatic or/and co-transcriptional capping.

54. A catalytic nucleic acid molecule, comprising
(i) a sequence selected from SEQ ID NO: 1-25,
(ii) a sequence having at least 80% identity with any one of SEQ ID NO: 1-25, and/or
(iii) a fragment of (i) and/or (ii),
wherein the catalytic nucleic acid molecule comprises a catalytic core.

55. The catalytic nucleic acid molecule of claim 54, wherein
Am is independently selected from A and 2′-O-methyladenosine,
Gm is independently selected from G and 2′-O-methylguanosine,
Um is independently selected from U and 2′-O-methyluridine, and/or
Cm is independently selected from C and 2′-O-methylcytidine.

56. The catalytic nucleic acid molecule of claims 54 and 55, which is an RNA molecule.

57. The catalytic nucleic acid molecule of any one of the claims 54-56, which is a ribozyme.

58. Use of the catalytic nucleic acid molecule of any one of the claims 54-57 in the method of any one of claims 1-52 for analyzing a population of RNA molecules, in a method for determining capping efficiency in a population of RNA molecules, in a method of analyzing an RNA molecule, and/or a method of capped RNA synthesis quality control.

59. A nucleic acid molecule, comprising
(i) a sequence of SEQ ID NO: 26 or SEQ ID NO:27,
(ii) a sequence having at least 90% identity with SEQ ID NO: 26 and/or SEQ ID NO:27.

60. The nucleic acid molecule of claim 59, which is an RNA molecule.

61. The nucleic acid molecule of claim 59 or 60, comprising a catalytic core of a ribozyme.


SEQUENCE LISTING (32 sequences)

SEQ ID NO: 1 - RNA, 39 nt, synthetic construct
  source 1..39: mol_type = other RNA
tgtgggctgatgaggccgtgaggccgaaaccagaagaat

SEQ ID NO: 2 - RNA, 45 nt, synthetic construct
  source 1..45: mol_type = other RNA
  modified_base 39: mod_base = um
  modified_base 40: mod_base = OTHER; note = 2'-O-methlyadenosine
  modified_base 41: mod_base = gm
  modified_base 42..43: mod_base = tm
  modified_base 44: mod_base = cm
  modified_base 45: mod_base = gm
ggggaccagaagaactgatgaggccgtgaggccgaaactagttcg

SEQ ID NO: 3 - RNA, 35 nt, synthetic construct
  source 1..35: mol_type = other RNA
tgtgggctgatgaggccgtgaggccgaaaccagaa

SEQ ID NO: 4 - RNA, 42 nt, synthetic construct
  source 1..42: mol_type = other RNA
agtctgtgggctgatgaggccgtgaggccgaaaccagaagaa

SEQ ID NO: 5 - RNA, 47 nt, synthetic construct
  source 1..47: mol_type = other RNA
  modified_base 28: mod_base = i
  modified_base 29..30: mod_base = um
  modified_base 31: mod_base = gm
  modified_base 32: mod_base = um
  modified_base 33: mod_base = gm
  modified_base 34..35: mod_base = um
  modified_base 36: mod_base = gm
  modified_base 37: mod_base = OTHER; note = 2'-O-methyladenosine
  modified_base 38: mod_base = gm
  modified_base 39: mod_base = OTHER; note = 2'-O-methyladenosine
  modified_base 40: mod_base = cm
  modified_base 41: mod_base = um
  modified_base 42: mod_base = OTHER; note = 2'-O-methyladenosine
  modified_base 43: mod_base = gm
  modified_base 44..46: mod_base = um
  modified_base 47: mod_base = OTHER; note = 2'-O-methyadenosine
gtatactgatgaggccgtgaggccgaanttgtgttgagactagttta

SEQ ID NO: 6 - RNA, 37 nt, synthetic construct
  source 1..37: mol_type = other RNA
  modified_base 31: mod_base = i
tctgagtcctgatgaggccgtgaggccgaantgggga

SEQ ID NO: 7 - RNA, 37 nt, synthetic construct
  source 1..37: mol_type = other RNA
  modified_base 30: mod_base = i
gagtctgctgatgaggccgtgaggccgaangggacca

SEQ ID NO: 8 - RNA, 37 nt, synthetic construct
  source 1..37: mol_type = other RNA
  modified_base 30: mod_base = i
gtctgtgctgatgaggccgtgaggccgaangaccaga

SEQ ID NO: 9 - RNA, 37 nt, synthetic construct
  source 1..37: mol_type = other RNA
  modified_base 30: mod_base = i
agtctgtctgatgaggccgtgaggccgaanggaccag

SEQ ID NO: 10 - RNA, 36 nt, synthetic construct
  source 1..36: mol_type = other RNA
  modified_base 29: mod_base = i
gtctgtctgatgaggccgtgaggccgaanggaccag

SEQ ID NO: 11 - RNA, 37 nt, synthetic construct
  source 1..37: mol_type = other RNA
  modified_base 30: mod_base = i
gaatactctgatgaggccgtgaggccgaantttattc

SEQ ID NO: 12 - RNA, 37 nt, synthetic construct
  source 1..37: mol_type = other RNA
  modified_base 30: mod_base = i
gaccagactgatgaggccgtgaggccgaanaatacta

SEQ ID NO: 13 - RNA, 43 nt, synthetic construct
  source 1..43: mol_type = other RNA
  modified_base 33: mod_base = i
ggggaccagactgatgaggccgtgaggccgaanaatactagtt

SEQ ID NO: 14 - RNA, 37 nt, synthetic construct
  source 1..37: mol_type = other RNA
  modified_base 30: mod_base = i
ggggaccctgatgaggccgtgaggccgaanaagaata

SEQ ID NO: 15 - RNA, 43 nt, synthetic construct
  source 1..43: mol_type = other RNA
  modified_base 33: mod_base = i
tgtggggaccctgatgaggccgtgaggccgaanaagaatacta

SEQ ID NO: 16 - RNA, 37 nt, synthetic construct
  source 1..37: mol_type = other RNA
  modified_base 30: mod_base = i
tctgtggctgatgaggccgtgaggccgaanaccagaa

SEQ ID NO: 17 - RNA, 43 nt, synthetic construct
  source 1..43: mol_type = other RNA
  modified_base 33: mod_base = i
gagtctgtggctgatgaggccgtgaggccgaanaccagaagaa

SEQ ID NO: 18 - RNA, 43 nt, synthetic construct
  source 1..43: mol_type = other RNA
  modified_base 33: mod_base = i
tgagtctgtgctgatgaggccgtgaggccgaangaccagaaga

SEQ ID NO: 19 - RNA, 43 nt, synthetic construct
  source 1..43: mol_type = other RNA
  modified_base 33: mod_base = i
ctgagtctgtctgatgaggccgtgaggccgaanggaccagaag

SEQ ID NO: 20 - RNA, 39 nt, synthetic construct
  source 1..39: mol_type = other RNA
  modified_base 29: mod_base = i
gtctgtctgatgaggccgtgaggccgaanggaccagaag

SEQ ID NO: 21 - RNA, 43 nt, synthetic construct
  source 1..43: mol_type = other RNA
  modified_base 33: mod_base = i
tctgagtctgctgatgaggccgtgaggccgaangggaccagaa

SEQ ID NO: 22 - RNA, 37 nt, synthetic construct
  source 1..37: mol_type = other RNA
  modified_base 30: mod_base = i
ctgagtcctgatgaggccgtgaggccgaantggggac

SEQ ID NO: 23 - RNA, 43 nt, synthetic construct
  source 1..43: mol_type = other RNA
  modified_base 33: mod_base = i
tctctgagtcctgatgaggccgtgaggccgaantggggaccag

SEQ ID NO: 24 - RNA, 37 nt, synthetic construct
  source 1..37: mol_type = other RNA
  modified_base 30: mod_base = i
ctctctgctgatgaggccgtgaggccgaantctgtgg

SEQ ID NO: 25 - RNA, 43 nt, synthetic construct
  source 1..43: mol_type = other RNA
  modified_base 33: mod_base = i
gttctctctgctgatgaggccgtgaggccgaantctgtgggga

SEQ ID NO: 26 - RNA, 23 nt, synthetic construct
  source 1..23: mol_type = other RNA
ctgatgaggccgtgaggccgaaa

SEQ ID NO: 27 - RNA, 23 nt, synthetic construct
  source 1..23: mol_type = other RNA
  modified_base 23: mod_base = i
ctgatgaggccgtgaggccgaan

SEQ ID NO: 28 - RNA, 25 nt, synthetic construct
  source 1..25: mol_type = other RNA
  modified_base 1: mod_base = m7g
  modified_base 2: mod_base = OTHER; note = 2'-O-methyladenosine
  modified_base 10: mod_base = m1f
  modified_base 13: mod_base = m1f
  modified_base 15..16: mod_base = m1f
  modified_base 18..19: mod_base = m1f
  modified_base 21: mod_base = m1f
  modified_base 24: mod_base = m1f
nnggcgaactagtattcttctggtc

SEQ ID NO: 29 - RNA, 25 nt, synthetic construct
  source 1..25: mol_type = other RNA
  modified_base 1: mod_base = m7g
  modified_base 10: mod_base = m1f
  modified_base 13: mod_base = m1f
  modified_base 15..16: mod_base = m1f
  modified_base 18..19: mod_base = m1f
  modified_base 21: mod_base = m1f
  modified_base 24: mod_base = m1f
naggcgaactagtattcttctggtc

SEQ ID NO: 30 - DNA, 16 bp, synthetic construct
  misc_feature 1..6: note = DNA
  source 1..16: mol_type = other DNA
  modified_base 7..8: mod_base = OTHER; note = 2'-O-methyladenosine
  modified_base 9: mod_base = gm
  modified_base 10..11: mod_base = OTHER; note = 2'-O-methyladenosine
  modified_base 12: mod_base = um
  modified_base 13: mod_base = OTHER; note = 2'-O-methyladenosine
  modified_base 14: mod_base = cm
  modified_base 15: mod_base = um
  modified_base 16: mod_base = OTHER; note = 2'-O-methyladenosine
  misc_feature 7..16: note = RNA
gaccagaagaatacta

SEQ ID NO: 31 - RNA, 20 nt, synthetic construct
  source 1..20: mol_type = other RNA
gcgaactagtattcttctgg

SEQ ID NO: 32 - RNA, 21 nt, synthetic construct
  source 1..21: mol_type = other RNA
gcgaactagtattcttctggt